SEQUENCE LISTING (213 sequences)

SEQ ID NO: 1 - DNA, 21 bp, Homo sapiens
acgggaagacaagttcatgta

SEQ ID NO: 2 - DNA, 21 bp, Homo sapiens
tcggcttctcctgaaagggaa

SEQ ID NO: 3 - DNA, 21 bp, Homo sapiens
atcgatagcatttgcagtata

SEQ ID NO: 4 - DNA, 21 bp, Homo sapiens
caggatcacactatcaagaaa

SEQ ID NO: 5 - DNA, 21 bp, Homo sapiens
cagaattgctagaacctttaa

SEQ ID NO: 6 - DNA, 21 bp, Homo sapiens
caccaacatggttgatttaaa

SEQ ID NO: 7 - DNA, 21 bp, Homo sapiens
cgccaacggcgcggcgatcaa

SEQ ID NO: 8 - DNA, 21 bp, Homo sapiens
acaggcgaacccgacgcagaa

SEQ ID NO: 9 - DNA, 21 bp, Homo sapiens
tcggctgggaccgttcatgta

SEQ ID NO: 10 - DNA, 21 bp, Homo sapiens
aaggtttatcccgccgataga

SEQ ID NO: 11 - DNA, 21 bp, Homo sapiens
caggagaattagatttctata

SEQ ID NO: 12 - DNA, 21 bp, Homo sapiens
ctggacctgtatatttgtaaa

SEQ ID NO: 13 - DNA, 21 bp, Homo sapiens
ctccgaggtctaaagtatata

SEQ ID NO: 14 - DNA, 21 bp, Homo sapiens
cacgggaactctccaaatatt

SEQ ID NO: 15 - DNA, 21 bp, Homo sapiens
aagatgaactttgcgaatgta

SEQ ID NO: 16 - DNA, 21 bp, Homo sapiens
gcggtgtttctcagaattgat

SEQ ID NO: 17 - DNA, 21 bp, Homo sapiens
ccagagctgagatgacttcaa

SEQ ID NO: 18 - DNA, 21 bp, Homo sapiens
ctcccggaaagcagaaaccaa

SEQ ID NO: 19 - DNA, 21 bp, Homo sapiens
ctgcggcagcgcaacactgaa

SEQ ID NO: 20 - DNA, 21 bp, Homo sapiens
caccgatctgcggcccatcta

SEQ ID NO: 21 - DNA, 21 bp, Homo sapiens
cacgagcaaggtggcggtgca

SEQ ID NO: 22 - DNA, 21 bp, Homo sapiens
caggaataatgcggtaggcaa

SEQ ID NO: 23 - DNA, 21 bp, Homo sapiens
cccgccagatctgaacagaaa

SEQ ID NO: 24 - DNA, 21 bp, Homo sapiens
caggttcgggccacgggtaaa

SEQ ID NO: 25 - DNA, 21 bp, Homo sapiens
gtggatgttgatggtagtaca

SEQ ID NO: 26 - DNA, 21 bp, Homo sapiens
cccagatcttagtaagctata

SEQ ID NO: 27 - DNA, 21 bp, Homo sapiens
aacagtggtcaatgtgcgaaa

SEQ ID NO: 28 - DNA, 21 bp, Homo sapiens
cagacgtctcccactgatgaa

SEQ ID NO: 29 - DNA, 21 bp, Homo sapiens
cacctatggccggataactaa

SEQ ID NO: 30 - DNA, 21 bp, Homo sapiens
ccggaatattgctacatacta

SEQ ID NO: 31 - DNA, 21 bp, Homo sapiens
aacaatttatgttcacattaa

SEQ ID NO: 32 - DNA, 21 bp, Homo sapiens
gagcgtctaatccgacagcaa

SEQ ID NO: 33 - DNA, 21 bp, Homo sapiens
aagtctaactatgaagtatta

SEQ ID NO: 34 - DNA, 21 bp, Homo sapiens
gacgatacagctaattcagaa

SEQ ID NO: 35 - DNA, 21 bp, Homo sapiens
gtggacgaatatgatccaaca

SEQ ID NO: 36 - DNA, 21 bp, Homo sapiens
cagacgtatattgtatcattt

SEQ ID NO: 37 - DNA, 21 bp, Homo sapiens
aaggagaatttaataaagata

SEQ ID NO: 38 - DNA, 21 bp, Homo sapiens
agggtgaaattcgtggagcaa

SEQ ID NO: 39 - DNA, 21 bp, Homo sapiens
ttcaatgttactgcactgaaa

SEQ ID NO: 40 - DNA, 21 bp, Homo sapiens
acagcacaacttcgagaacaa

SEQ ID NO: 41 - DNA, 21 bp, Homo sapiens
aagggtcttactgaagtaaca

SEQ ID NO: 42 - DNA, 21 bp, Homo sapiens
tacccagataccgatgttata

SEQ ID NO: 43 - DNA, 21 bp, Homo sapiens
atgattattaacgatgtccaa

SEQ ID NO: 44 - DNA, 21 bp, Homo sapiens
ttcggaatgatgagcacacaa

SEQ ID NO: 45 - DNA, 21 bp, Homo sapiens
taccttatagttactgtgtaa

SEQ ID NO: 46 - DNA, 21 bp, Homo sapiens
cacgaccatctttcacgaata

SEQ ID NO: 47 - DNA, 21 bp, Homo sapiens
ctggacggccgtgttcgagta

SEQ ID NO: 48 - DNA, 21 bp, Homo sapiens
accatagagaatgttcgccaa

SEQ ID NO: 49 - DNA, 21 bp, Homo sapiens
ggccttggaatctatagtata

SEQ ID NO: 50 - DNA, 21 bp, Homo sapiens
tacggtagtgatgaaattata

SEQ ID NO: 51 - DNA, 21 bp, Homo sapiens
ctcatcaatgctctcggtcaa

SEQ ID NO: 52 - DNA, 21 bp, Homo sapiens
accctgagtcctgccatctaa

SEQ ID NO: 53 - DNA, 21 bp, Homo sapiens
caaggtcgtaaatgcatgcta

SEQ ID NO: 54 - DNA, 21 bp, Homo sapiens
ctgggcctgaacataatttca

SEQ ID NO: 55 - DNA, 21 bp, Homo sapiens
cacctccatcctatgaagaga

SEQ ID NO: 56 - DNA, 21 bp, Homo sapiens
cactagtgtctcagaccagaa

SEQ ID NO: 57 - DNA, 21 bp, Homo sapiens
caaggtcacttcgggcagaaa

SEQ ID NO: 58 - DNA, 21 bp, Homo sapiens
ctcgggcacgctcaagacgta

SEQ ID NO: 59 - DNA, 21 bp, Homo sapiens
aagacggtgtatcgagggcta

SEQ ID NO: 60 - DNA, 21 bp, Homo sapiens
caggtagacctgtttggtata

SEQ ID NO: 61 - DNA, 21 bp, Homo sapiens
aagctcgagtgtctcggcaaa

SEQ ID NO: 62 - DNA, 21 bp, Homo sapiens
aaggagacaactaaactgcaa

SEQ ID NO: 63 - DNA, 21 bp, Homo sapiens
atggaccaatgccagggttaa

SEQ ID NO: 64 - DNA, 21 bp, Homo sapiens
ctgcattaaagtgctaatgat

SEQ ID NO: 65 - DNA, 21 bp, Homo sapiens
agcaagtgagttgcccattaa

SEQ ID NO: 66 - DNA, 21 bp, Homo sapiens
cacctattgaccatactacaa

SEQ ID NO: 67 - DNA, 21 bp, Homo sapiens
ttggtgcctttgaacgctcaa

SEQ ID NO: 68 - DNA, 21 bp, Homo sapiens
cgggaagtgaggagatttcta

SEQ ID NO: 69 - DNA, 21 bp, Homo sapiens
cggcgctaaggtggccatcaa

SEQ ID NO: 70 - DNA, 21 bp, Homo sapiens
cacaggcaagacgctgttcaa

SEQ ID NO: 71 - DNA, 21 bp, Homo sapiens
ctgggaggtgcgcgccgtgta

SEQ ID NO: 72 - DNA, 21 bp, Homo sapiens
caggactcacgtagcattaaa

SEQ ID NO: 73 - DNA, 21 bp, Homo sapiens
cccgcttggatcagtgaccat

SEQ ID NO: 74 - DNA, 21 bp, Homo sapiens
cccgtgtgtgttggaagggaa

SEQ ID NO: 75 - DNA, 21 bp, Homo sapiens
acgcagcgagagaataaagaa

SEQ ID NO: 76 - DNA, 21 bp, Homo sapiens
cagagactgtactacgacaaa

SEQ ID NO: 77 - DNA, 21 bp, Homo sapiens
aagaagcaagtggctatcata

SEQ ID NO: 78 - DNA, 21 bp, Homo sapiens
caccgggatagatagaactat

SEQ ID NO: 79 - DNA, 21 bp, Homo sapiens
aaggagcttattgagctatca

SEQ ID NO: 80 - DNA, 21 bp, Homo sapiens
accgtttgggatggttgatta

SEQ ID NO: 81 - DNA, 21 bp, Homo sapiens
cggcccagcgttcaagcccaa

SEQ ID NO: 82 - DNA, 21 bp, Homo sapiens
cggcaggactcttatcttaaa

SEQ ID NO: 83 - DNA, 21 bp, Homo sapiens
ttcgatggtttcgatcacaaa

SEQ ID NO: 84 - DNA, 21 bp, Homo sapiens
caggtaaatgtcaaggcacaa

SEQ ID NO: 85 - DNA, 21 bp, Homo sapiens
ctgcactgggaaggaaacaaa

SEQ ID NO: 86 - DNA, 21 bp, Homo sapiens
cacagaagcggtggaagccaa

SEQ ID NO: 87 - DNA, 21 bp, Homo sapiens
caggtgagcgacgaccttaca

SEQ ID NO: 88 - DNA, 21 bp, Homo sapiens
ttgcgactagaggttaaacta

SEQ ID NO: 89 - DNA, 21 bp, Homo sapiens
aacagtcagtatgcaatctta

SEQ ID NO: 90 - DNA, 21 bp, Homo sapiens
cgggctgctaatattcttgta

SEQ ID NO: 91 - DNA, 21 bp, Homo sapiens
aaggctcataatctatcaaga

SEQ ID NO: 92 - DNA, 21 bp, Homo sapiens
cagtaggcacttaagcattta

SEQ ID NO: 93 - DNA, 21 bp, Homo sapiens
cgcggctggagtcatgctata

SEQ ID NO: 94 - DNA, 21 bp, Homo sapiens
caagaacatgatgttattaaa

SEQ ID NO: 95 - DNA, 21 bp, Homo sapiens
cactacaaacacaatattcaa

SEQ ID NO: 96 - DNA, 21 bp, Homo sapiens
ctgagacagtttgctaattta

SEQ ID NO: 97 - DNA, 21 bp, Homo sapiens
atcagtgaaatgacagataaa

SEQ ID NO: 98 - DNA, 21 bp, Homo sapiens
tgccttgtacataatactatt

SEQ ID NO: 99 - DNA, 21 bp, Homo sapiens
tagccgctcatcttcgatgaa

SEQ ID NO: 100 - DNA, 21 bp, Homo sapiens
ctggttcaacttccacggtca

SEQ ID NO: 101 - DNA, 21 bp, Homo sapiens
gaggactactatggaccttca

SEQ ID NO: 102 - DNA, 21 bp, Homo sapiens
cacgtttggaaagaatattaa

SEQ ID NO: 103 - DNA, 21 bp, Homo sapiens
cccaaatgtcaaactaccaaa

SEQ ID NO: 104 - DNA, 21 bp, Homo sapiens
ttgggctatagcacacagtta

SEQ ID NO: 105 - DNA, 21 bp, Homo sapiens
cacctacaactcagcacatga

SEQ ID NO: 106 - DNA, 21 bp, Homo sapiens
ctggaatttgatattcattga

SEQ ID NO: 107 - DNA, 21 bp, Homo sapiens
aagcatttgtttgtacaagat

SEQ ID NO: 108 - DNA, 21 bp, Homo sapiens
aactttgatgtgattaaggga

SEQ ID NO: 109 - DNA, 21 bp, Homo sapiens
caggagagaattagtcgatat

SEQ ID NO: 110 - DNA, 21 bp, Homo sapiens
ctgcaattctacagtttctta

SEQ ID NO: 111 - DNA, 21 bp, Homo sapiens
cagcgagctgaccgagctcaa

SEQ ID NO: 112 - DNA, 21 bp, Homo sapiens
taccctattacagagagtcaa

SEQ ID NO: 113 - DNA, 21 bp, Homo sapiens
ccctgtagaggtggtattaaa

SEQ ID NO: 114 - DNA, 21 bp, Homo sapiens
tcaagttggcctacaatgaaa

SEQ ID NO: 115 - DNA, 21 bp, Homo sapiens
tagcagcatgtcctacggaaa

SEQ ID NO: 116 - DNA, 21 bp, Homo sapiens
aaggcgatagatgatgaggaa

SEQ ID NO: 117 - DNA, 21 bp, Homo sapiens
gagatcgaagaagattataaa

SEQ ID NO: 118 - DNA, 21 bp, Homo sapiens
cagaaggagcctgtccctcaa

SEQ ID NO: 119 - DNA, 21 bp, Homo sapiens
caggacctatctggaagagga

SEQ ID NO: 120 - DNA, 21 bp, Homo sapiens
gccctgctgcttcctaactta

SEQ ID NO: 121 - DNA, 21 bp, Homo sapiens
gcgggcaggtgtgcatatcaa

SEQ ID NO: 122 - DNA, 21 bp, Homo sapiens
ccggatcacattacatggctt

SEQ ID NO: 123 - DNA, 21 bp, Homo sapiens
cagctgcatggccaacacgaa

SEQ ID NO: 124 - DNA, 21 bp, Homo sapiens
tgggccaacaaagaacactaa

SEQ ID NO: 125 - DNA, 21 bp, Homo sapiens
cagggtctacgtctacctgca

SEQ ID NO: 126 - DNA, 21 bp, Homo sapiens
aaagggagtcattaacaacta

SEQ ID NO: 127 - DNA, 21 bp, Homo sapiens
cacggtaccaatggcattcat

SEQ ID NO: 128 - DNA, 21 bp, Homo sapiens
atcgaatttgtcaaccaatat

SEQ ID NO: 129 - DNA, 21 bp, Homo sapiens
ccgggacttctgtgacgtcca

SEQ ID NO: 130 - DNA, 21 bp, Homo sapiens
aaagatagtcgtcaaggtcaa

SEQ ID NO: 131 - DNA, 21 bp, Homo sapiens
cccaatcgtctgtactatgaa

SEQ ID NO: 132 - DNA, 21 bp, Homo sapiens
ttggaggtgttaaatctacaa

SEQ ID NO: 133 - DNA, 21 bp, Homo sapiens
tgcgcagacgaaagaagaata

SEQ ID NO: 134 - DNA, 21 bp, Homo sapiens
ccgggaagccgcctataccaa

SEQ ID NO: 135 - DNA, 21 bp, Homo sapiens
caggaacaacagcatctttca

SEQ ID NO: 136 - DNA, 21 bp, Homo sapiens
caaggacttgccaacattcaa

SEQ ID NO: 137 - DNA, 21 bp, Homo sapiens
cacgcgtttcctcaccaccta

SEQ ID NO: 138 - DNA, 21 bp, Homo sapiens
ccctgggaacaagacccacaa

SEQ ID NO: 139 - DNA, 21 bp, Homo sapiens
cagcagaagtgggtgcaggaa

SEQ ID NO: 140 - DNA, 21 bp, Homo sapiens
aacgctaaggtgggtagccta

SEQ ID NO: 141 - DNA, 21 bp, Homo sapiens
cagaaatgcatgggtaaggta

SEQ ID NO: 142 - DNA, 21 bp, Homo sapiens
cagcaggatgagaccaaacaa

SEQ ID NO: 143 - DNA, 21 bp, Homo sapiens
ccggctcaggagcaagatgaa

SEQ ID NO: 144 - DNA, 21 bp, Homo sapiens
tccataagtgactgtgctaaa

SEQ ID NO: 145 - DNA, 21 bp, Homo sapiens
gagcataggcgtcgtaatgta

SEQ ID NO: 146 - DNA, 21 bp, Homo sapiens
aagggtcatgtaggtacaaca

SEQ ID NO: 147 - DNA, 21 bp, Homo sapiens
ctcccttattcaggtggacaa

SEQ ID NO: 148 - DNA, 21 bp, Homo sapiens
ttgcagtactgtatgctcaaa

SEQ ID NO: 149 - DNA, 21 bp, Homo sapiens
ctgctgaatgttgccatatta

SEQ ID NO: 150 - DNA, 21 bp, Homo sapiens
cccacgaggagtggacaatat

SEQ ID NO: 151 - DNA, 21 bp, Homo sapiens
aaggctggttgtggcctacaa

SEQ ID NO: 152 - DNA, 21 bp, Homo sapiens
aagtacgactgtagttatcta

SEQ ID NO: 153 - DNA, 21 bp, Homo sapiens
tcccatgggattattctctat

SEQ ID NO: 154 - DNA, 21 bp, Homo sapiens
caccggcattgaggtagtgat

SEQ ID NO: 155 - DNA, 21 bp, Homo sapiens
caggccgcctttgagtcttta

SEQ ID NO: 156 - DNA, 21 bp, Homo sapiens
ctgggcatgaggaggtttaaa

SEQ ID NO: 157 - DNA, 21 bp, Homo sapiens
cgggatcactttggaggacta

SEQ ID NO: 158 - DNA, 21 bp, Homo sapiens
caacagtttgttcctcgaata

SEQ ID NO: 159 - DNA, 21 bp, Homo sapiens
tgggcaaacagtagacttata

SEQ ID NO: 160 - DNA, 21 bp, Homo sapiens
aagcatgaaactttaccctat

SEQ ID NO: 161 - DNA, 21 bp, Homo sapiens
ctggctgttaggagtaaatca

SEQ ID NO: 162 - DNA, 21 bp, Homo sapiens
tagcaatttggtacctatgta

SEQ ID NO: 163 - DNA, 21 bp, Homo sapiens
aagcatttaattcacagtcaa

SEQ ID NO: 164 - DNA, 21 bp, Homo sapiens
ttggataattctatacataaa

SEQ ID NO: 165 - DNA, 21 bp, Homo sapiens
cagggtgatgtgcatgctgaa

SEQ ID NO: 166 - DNA, 21 bp, Homo sapiens
cagcaggaagaagtagctaaa

SEQ ID NO: 167 - DNA, 21 bp, Homo sapiens
cagcagaaagctattctggaa

SEQ ID NO: 168 - DNA, 21 bp, Homo sapiens
tcccaaggcaccagtactcta

SEQ ID NO: 169 - DNA, 21 bp, Homo sapiens
ttggaccttcacatatctaaa

SEQ ID NO: 170 - DNA, 21 bp, Homo sapiens
aacagagaacttgagtgtatt

SEQ ID NO: 171 - DNA, 21 bp, Homo sapiens
aagaatttcctttatgatata

SEQ ID NO: 172 - DNA, 21 bp, Homo sapiens
cccggcgaggagaacggcaaa

SEQ ID NO: 173 - DNA, 21 bp, Homo sapiens
cccacgaagtgttggatataa

SEQ ID NO: 174 - DNA, 21 bp, Homo sapiens
ccacagcactattgagtgaaa

SEQ ID NO: 175 - DNA, 21 bp, Homo sapiens
cagcagcagccagaaacgata

SEQ ID NO: 176 - DNA, 21 bp, Homo sapiens
caggcgggcgtcgacgtcata

SEQ ID NO: 177 - DNA, 21 bp, Homo sapiens
tgggatccggctaaagaaata

SEQ ID NO: 178 - DNA, 21 bp, Homo sapiens
aatgtgggtcatattgcgaaa

SEQ ID NO: 179 - DNA, 21 bp, Homo sapiens
ctggagaagttaaagcctgaa

SEQ ID NO: 180 - DNA, 21 bp, Homo sapiens
gaggaggtcgttcagattcta

SEQ ID NO: 181 - DNA, 21 bp, Homo sapiens
ttccgccttgttggtctgaaa

SEQ ID NO: 182 - DNA, 21 bp, Homo sapiens
tccgaagatcttctcaaggaa

SEQ ID NO: 183 - DNA, 21 bp, Homo sapiens
cagcactacattgacctgaaa

SEQ ID NO: 184 - DNA, 21 bp, Homo sapiens
tagagcatatttgccaataaa

SEQ ID NO: 185 - DNA, 21 bp, Homo sapiens
tgggctggtgaagtacatgaa

SEQ ID NO: 186 - DNA, 21 bp, Homo sapiens
caggttggcaggaacatcatt

SEQ ID NO: 187 - DNA, 21 bp, Homo sapiens
cctggtgggcaagatcatcaa

SEQ ID NO: 188 - DNA, 21 bp, Homo sapiens
ctgcatcgaggttggcaagaa

SEQ ID NO: 189 - DNA, 21 bp, Homo sapiens
acggccgccttgtcaagtata

SEQ ID NO: 190 - DNA, 21 bp, Homo sapiens
acccaactgaatgaagagcta

SEQ ID NO: 191 - DNA, 21 bp, Homo sapiens
cagctcagccctgtacatata

SEQ ID NO: 192 - DNA, 21 bp, Homo sapiens
atggaaggtcctgacagatta

SEQ ID NO: 193 - DNA, 21 bp, Homo sapiens
aacattgtaccctatactata

SEQ ID NO: 194 - DNA, 21 bp, Homo sapiens
acagagtgctataactataaa

SEQ ID NO: 195 - DNA, 21 bp, Homo sapiens
acacagagtgctataactata

SEQ ID NO: 196 - DNA, 21 bp, Homo sapiens
accgctttgtacatgagaata

SEQ ID NO: 197 - DNA, 21 bp, Homo sapiens
atcgcggagctgatctacaaa

SEQ ID NO: 198 - DNA, 21 bp, Homo sapiens
ttcgatcttgtctccaattaa

SEQ ID NO: 199 - DNA, 21 bp, Homo sapiens
atgggaagtcgtgtcatccta

SEQ ID NO: 200 - DNA, 21 bp, Homo sapiens
tacccactaatggacaatcaa

SEQ ID NO: 201 - DNA, 21 bp, Homo sapiens
caggtagttaagttagggtta

SEQ ID NO: 202 - DNA, 21 bp, Homo sapiens
cacgctgctcatgaccggcaa

SEQ ID NO: 203 - DNA, 21 bp, Homo sapiens
gcccaccacatttgacatcta

SEQ ID NO: 204 - DNA, 21 bp, Homo sapiens
cacggttagatcatatctgaa

SEQ ID NO: 205 - DNA, 21 bp, Homo sapiens
aaggtcgtcctttctgaacaa

SEQ ID NO: 206 - DNA, 21 bp, Homo sapiens
ctccgtcagcctcaccatgaa

SEQ ID NO: 207 - DNA, 21 bp, Homo sapiens
gacgacatccctggaatggaa

SEQ ID NO: 208 - DNA, 21 bp, Homo sapiens
cacaagattcatcgtgattca

SEQ ID NO: 209 - DNA, 21 bp, Homo sapiens
ttggaagccattacgcataaa

SEQ ID NO: 210 - DNA, 21 bp, Homo sapiens
cacagagatgttatttatgca

SEQ ID NO: 211 - DNA, 21 bp, Homo sapiens
cagtacgagatccttatggaa

SEQ ID NO: 212 - DNA, 21 bp, Homo sapiens
aggcgacaactttcaagtata

SEQ ID NO: 213 - DNA, 21 bp, Homo sapiens
aaccctgtatagaattaatta

(19) United States
(12) Patent Application Publication
(10) Pub. No.: US 2013/0116302 A1
(43) Pub. Date: May 9, 2013

(54) PHARMACEUTICAL COMPOSITION FOR THE TREATMENT OF CHLAMYDIAL INFECTION

(71) Applicants: Rajendra Kumar Gurumurthy, Berlin (DE); Andre Paul Mauerer, Berlin (DE); Thomas F. Meyer, Berlin (DE); Marion Rother, Berlin (DE); Nikolaus Machuy, Hamburg (DE); Erik Gonzalez Martinez, Berlin (DE)

(72) Inventors: Rajendra Kumar Gurumurthy, Berlin (DE); Andre Paul Mauerer, Berlin (DE); Thomas F. Meyer, Berlin (DE); Marion Rother, Berlin (DE); Nikolaus Machuy, Hamburg (DE); Erik Gonzalez Martinez, Berlin (DE)

(73) Assignee: Max-Planck-Gesellschaft zur Foerderung der Wissenschaften e.V., Muenchen (DE)

(21) Appl. No.: 13/634,312

(22) PCT Filed: Mar. 11, 2011
(86) PCT No.: PCT/EP2011/053716

§ 371 Date: Jan. 17, 2013

(30) Foreign Application Priority Data
Mar. 12, 2010 (EP) 10156422.7

(51) Int. Cl. A61K 31/713

(52) U.S. Cl. CPC A61K 31/713
USPC 514/44 A; 536/24.5

(57) ABSTRACT

Subject of the present invention is a pharmaceutical composition comprising at least one inhibitor of a microorganism selected from the family Chlamydiaceae.

Subject of the present invention is a pharmaceutical composition comprising at least one inhibitor of a microorganism selected from the family Chlamydiaceae, optionally together with pharmaceutically acceptable carriers, adjuvants, diluents or/and additives, wherein the inhibitor is selected from compounds capable of modulating the activity of a polypeptide selected from Table 1. Another subject of the present invention is screening method for identification of a compound suitable as inhibitor in a pharmaceutical composition defined herein, comprising the steps: (a) providing a eukaryotic host cell or/and a transgenic non-human animal capable of being infected with a microorganism selected from the family Chlamydiaceae, such as Chlamydia, in particular Chlamydia trachomatis, (b) contacting the cell or/and the transgenic animal of (a) with a microorganism selected from the family Chlamydiaceae, such as Chlamydia, in particular Chlamydia trachomatis, and contacting a compound with the cell or/and the transgenic non-human animal of (a), and (c) selecting a compound which inhibits the microorganism of (a).

Chlamydiae are Gram-negative, obligate, intracellular bacterial pathogens and the causative agents of a wide range of human and animal diseases. Chlamydia trachomatis (Ctr) is a human pathogen associated with several diseases, including sexually transmitted diseases (Brunham and Rey-Ladino, 2005) and preventable blindness (trachoma) (Wright et al., 2008). The developmental cycle of Ctr alternates between two functionally and morphologically distinct forms: the extracellular, infectious, metabolically inactive elementary body (EB) and the intracellular, metabolically active, replicating reticulate body (RB). EBs infect host cells and differentiate into RBs within a membrane-bound, protective vacuole called the inclusion. RBs multiply, and at the end of the cycle they redifferentiate into EBs, which are released from cells to initiate a new developmental cycle by infecting neighboring cells (Moulder, 1991).

Acivicin (L-[αS,5S]-α-amino-3-chloro-4,5-dihydro-5-isoxazoleacetic acid) irreversibly inhibits the γ-glutamine amidotransferase activity of GMPS (Chittur et al., 2001). Acivicin is an α-amino acid produced by Streptomyces sviceus that contains the dihydroisoxazole ring as a mimic of the glutamine γ-carboxiamide group. Acivicin has been classified along with DON (6-diazo-5-oxo-L-norleucine) and azaserine as affinity analogues of glutamine amidotransferases (GATs) (O'Dwyer et al., 1984).

Acivicin inhibits each of the four amidotransferases of the novo pathway of purine and pyrimidine synthesis: phosphoribosyl pyrophosphate amidotransferase (PPAT), guanosine monophosphate synthase (GMPS), carbamoyl-phosphate synthetase 2, aspartate transcarbamylase, and dihydroorotase (CAD), and UTP-ammonia ligase 1 (CTPS). The inhibition of these enzymes result in decrease of cellular UTP, CTP, and GMP concentrations, with no alteration in ATP or ITP pools (Neil et al., 1979).

The effect of acivicin on eukaryotic parasite growth has been investigated:
kills both the vector and the host form of Leishmania donovani (Mukherjee et al., 1990). Mukherjee at al. investigated acivicin in the context of inhibiting the carbamyl phosphate synthetase II, the first enzyme of the pyrimidine biosynthetic pathway.
has been shown to inhibit the growth of P. falciparum in vitro (Vilmont et al., 1990).
its CTPS inhibitory activity has been correlated to the observed antitrypanosomal activity against bloodstream T. brucei in culture and in a mouse model (Hofer et al., 2001, Fijolek et al. 2007).

There is one report of acivicin use in bacteria. Orth, R. et al. (2010) report the synthesis of acivicin inspired 3-chloro- and 3-bromo-dihydroisoxazole probes and their application in target profiling in non-pathogenic and as well as in pathogenic bacteria such as S. aureus and multiresistant S. aureus (MRSA).

Weber and others (1991) have demonstrated that in hepatoma and several other tumors, derived from experimental and human sources, the rate-limiting enzymes of nucleic acid biosynthesis show markedly increased activity.

The silencing of gene expression by RNA interference (RNAi) technology is proving to be a powerful tool to investigate the function of host proteins. Here, we present a systematic siRNA-based loss-of-function screen aimed at discovering host cell factors that interfere with the entry, survival, and replication of Ctr within human epithelial cells. We identified 59 host cell factors whose knockdown altered Ctr infectivity (see Table 1a). These factors included K-Ras and Raf-1, which when knocked down led to the increased growth of Ctr. Despite the depletion of K-Ras and Raf-1, ERK was still activated after the infection of cells with Ctr, which was accompanied by the strong stimulation of cPLA₂. This suggested that activation of ERK in Ctr-infected cells occurred through a K-Ras- and Raf-1-independent mechanism. Infection by Ctr also led to the Akt1- and Akt2-dependent phosphorylation of Raf-1 at Ser²⁵⁹, a modification known to inactivate Raf-1 (Rommel et al., 1996; Zimmermann and Moelling, 1999). In addition, we showed that Raf-1 was recruited to the inclusion in an Akt- and 14-3-3β-dependent manner. These data suggest that infection with Ctr triggers a modular regulation of components of the Ras-Raf-MEK-ERK pathway to support growth of the pathogen.

In the present invention, by modulation of a polypeptide selected from Table 1, Akt1, Akt2, Akt and 14-3-3β, a chlamydial infection can be successfully treated. A polypeptide selected from Table 1, Akt1, Akt2, Akt and 14-3-3β is a suitable target for the prophylaxis or/and treatment of an infection with a microorganism selected from the family Chlamydiaceae. A polypeptide selected from Table 1, Akt1, Akt2, Akt and 14-3-3β may be used in a screening method, as described herein, for compounds suitable for the prophylaxis or/and treatment of an infection with a microorganism selected from the family Chlamydiaceae. Furthermore, a modulator of a polypeptide selected from Table 1, Akt1, Akt2, Akt and 14-3-3β may be used for the prophylaxis or/and treatment of an infection with a microorganism selected from the family Chlamydiaceae. The subject-matter of the present invention is further described by the claims disclosed herein.

A preferred embodiment of the present invention refers to guanosine monophosphate synthase GMPS.

In the present invention, modulation of the GMPS is in particular modulation of the activity of GMPS. Modulation of the GMPS refers in particular to the modulation of GMP synthesis by the GMPS. In the present invention, inhibition of the GMPS is in particular inhibition of the activity of GMPS. Inhibition of the GMPS refers in particular to the inhibition of GMP synthesis by the GMPS.

Modulation of GMPS includes modulation of the interaction of GMPS with HAUSP, such as inhibition of the interaction of GMPS with HAUSP. Modulation of GMPS also includes modulation of recruitment of GMPS to the chlamydial inclusion, such as inhibition of recruitment of GMPS to the chlamydial inclusion.

Another preferred embodiment of the present invention refers to Akt1, Akt2, or/and Akt.

Yet another preferred embodiment of the present invention refers to 14-3-3β.

In the present invention, a reference to Table 1 includes a reference to Table 1a and Table 1b.

Specific embodiments of the present invention refer to the specific nucleic acid sequences, the specific polypeptide sequences, and the specific targets disclosed in Table 1. Preferred embodiments refer to the specific targets disclosed in Table 1. In the present invention, a “target” is a target for a modulator for the prevention or/and treatment of a chlamydial infection. A “target”, as used herein, includes a nucleic acid describing a gene, or/and a polypeptide encoded by said gene. Table 1 discloses target nucleic acid sequences and target polypeptide sequences. A target nucleotide sequence can comprise the complete sequence of a gene, or a partial sequence thereof, such as an siRNA target sequence. In Table 1, target nucleic acid sequences and target polypeptide sequences are described for example by at least one selected from NCBI gene symbol, Entrez Gene Id, mRNA accession number, and EC number.

In the present invention, “modulation” includes inhibition and activation.

If not stated otherwise, fragments of polypeptides or partial sequences of polypeptides, as used herein, may have a length of at least 10 amino acid residues, at least 20 amino acid residues, at least 30 amino acid residues, at least 40 amino acid residues, at least 50 amino acid residues, at least 80 amino acid residues, at least 100 amino acid residues, or at least 150 amino acid residues, up to the total length of the polypeptide.

If not stated otherwise, fragments of nucleic acid molecules or partial sequences of nucleic acid molecules, as used herein, may have a length of at least 15 nucleic acid residues, at least 30 nucleic acid residues, at least 60 nucleic acid residues, at least 90 nucleic acid residues, at least 120 nucleic acid residues, at least 150 nucleic acid residues, at least 200 nucleic acid residues, at least 240 nucleic acid residues, at least 300 nucleic acid residues, or at least 450 nucleic acid residues, up to the total length of the nucleic acid molecule.

The invention is further illustrated by the following Figures and Examples.

### FIGURE LEGENDS

FIG. 1: A loss-of-function screen for host factors involved in the development cycle of Chlamydia (Ctr). (A) The development cycle of Ctr. Ctr EBs (green) enter the host cell (step 1) and differentiate to RBs (red) (steps 1 and 2). The RBs multiply (step 3) and redifferentiate back to EBs (step 4) that can infect new host cells. (B) Cells were seeded (step 1) and transfected (step 2) in triplicate. At 72 hours post-transfection, one plate was fixed (step 3) to monitor any specific effects of the siRNAs used on cell growth. The remaining two plates were infected with Ctr (step 4), and at 24 hours post-infection one plate was fixed to evaluate the number and size of Ctr infectious particles (infection, step 5). Fresh cells were seeded (step 6) and infected with the lysate from the second infected plate at 48 hours post-infection (steps 7 and 8), which were fixed 24 hours later to measure infectivity (step 9). Nuclei in the host cells of all of the plates were stained with Hoechst and Chlamydia were detected with an antibody against Ctr (step 10). Images were acquired (step 11) and subjected to image and data analysis (steps 12 and 13). (C) The siRNAs siLuci, siARF1, and siLC3 were established as having no effect, an activating effect, or an inhibitory effect, respectively, on infectivity of Ctr from transfected cells. Shown are representative images and the normalized infectivity rates±standard deviation (SD) of three independent experiments. siLuci was used as a reference control.

FIG. 2: Identification and validation of hits from the primary screen. (A) Infectivity data of cells transfected with a pool of two siRNAs per gene were analyzed in parallel by two statistical normalization methods: B-Score and percent of control (POC). siLC3 inhibitory controls are marked in green, siARF1 activating controls in red and samples in black. The black lines indicate the defined thresholds used for defining the primary hits. All of the 80 overlapping primary hits from both statistical analysis methods and the 26 non-overlapping primary hits that were identified exclusively with the B-Score and POC methods were chosen for further validation. (B) Validation of the hits was performed for 132 primary hits with four independent siRNAs per gene. Data were analyzed by POC normalization and validated hits were grouped into “strong,” “medium,” and “weak” hits according to the number of siRNAs eliciting the biological effect. (C) Validated hits are grouped according to the used statistical analysis for the definition of primary hits. The numbers of scored hits from each of the methods of analysis used in the primary screen as well as the overlapping genes are shown.

FIG. 3: Activation of ERK after infection with Ctr is independent of KRas and Raf-1. Western blotting analysis (30 hours post-infection) of (A) uninfected and Ctr-infected cells with and without U0126, (B) uninfected and Ctr-infected cells transfected with siRNAs targeting luciferase or MEK1 and 2, and (C) uninfected and Ctr-infected cells transfected with siRNAs targeting luciferase, K-Ras, or Raf-1. β-actin was used as a loading control. (D) Uninfected and Ctr-infected cells treated with or without UO126 were lysed at 48 hours post-infection and these samples were used to infect freshly seeded cells. These cells were fixed 24 hours post-infection and the infectivity was quantified. Data shown are the mean±SD of three experiments normalized to mock-treated, infected cells. (E) Uninfected and Ctr-infected cells, untransfected of transfected with siRNAs specific for luciferase, K-Ras, or Raf-1 were lysed 48 hours post-infection and these samples were then used to infect freshly seeded cells. These cells were fixed 24 hours post-infection and the infectivity of the bacteria was quantified. Data shown are the mean±SD of three independent experiments normalized to infected and siLuci transfected cells. Western blots depicted in (A) to (C) are representative of three experiments. For knockdown of K-Ras and Raf-1, two independent siRNAs were used (indicated as 1 and 2).

FIG. 4: Phosphorylation of Raf-1 at Ser259 after infection with Ctr depends on Akt. Uninfected and Ctr-infected HeLa cells transfected with siRNAs specific for luciferase or Akt (siAkt1+2) were harvested 30 hours post-infection and subjected to Western blotting analysis for the detection of Akt, pERK, Raf-1, and pRaf-1 (Ser259). β-actin was used as a loading control. One blot representative of three independent experiments is shown.

FIG. 5: Translocation of Raf-1 to the Ctr inclusion is dependent on its phosphorylation at Ser259. Uninfected and Ctr-infected HeLa cells were fixed 30 hours post-infection and were incubated with antibodies against 14-3-3β and Raf-1 (A) or against 14-3-3β and pRaf-1 at Ser259 (B). Images were acquired with a confocal microscope. Overlaid images show the colocalization of 14-3-3β and Raf-1 with the Chlamydia inclusion. Uninfected and Ctr-infected HeLa cells transfected with plasmids encoding wild-type (WT) Raf-1 (C) or the S259A mutant of Raf-1 (D) were fixed 30 hours post-infection and incubated with an antibody against the HA tag. Images were acquired with a fluorescence microscope. Chlamydial inclusions are marked with an asterisk. Overlaid images show the translocation of WT, but not mutant, Raf-1 to the inclusion. Images are representative of three independent experiments.

FIG. 6: Translocation of Raf-1 to the inclusion is dependent on Akt and on a direct interaction with 14-3-3β. (A to C) Uninfected and Ctr-infected HeLa cells transfected with siRNAs specific for luciferase (A), Akt1/2 (B), or 14-3-36 (C) were lysed 30 hours post-infection, separated into subcellular fractions, and subjected to Western blotting analysis for the presence of Raf-1 and chlamydial Hsp60. Calpain, LAMP-1, lamin-A/C, and cytokeratin-8 were used as markers for cytosolic, membrane-organelle, nuclear, and cytoskeletal subcellular fractions, respectively. Blots shown are representative of three independent experiments. (D) Uninfected and Ctr-infected HeLa cells were stained with the Duolink in situ PLA kit with antibodies against Raf-1 and 14-3-3β. Fluorescent dots represent interactions between Raf-1 and 14-3-3β. Antibodies against MEK1/2 and 14-3-3β were used as negative controls. Inclusions are marked with an asterisk. Images shown are representative of three independent experiments.

Table 1: (a) Results of the screening for genes or/and polypeptides involved in chlamydial infection, (b) Results of the screening for genes or/and polypeptides involved in host cell nucleotide metabolism, which genes or/and polypeptides are essential for Chlamydia growth, propagation or/and infection.

### EXAMPLE 1

### A Loss-of-Function Screen Reveals Ras- and Raf-Independent MEK-ERK Signaling During Chlamydia trachomatis Infection

### Results

### 1) Screen

1a) Primary Screen

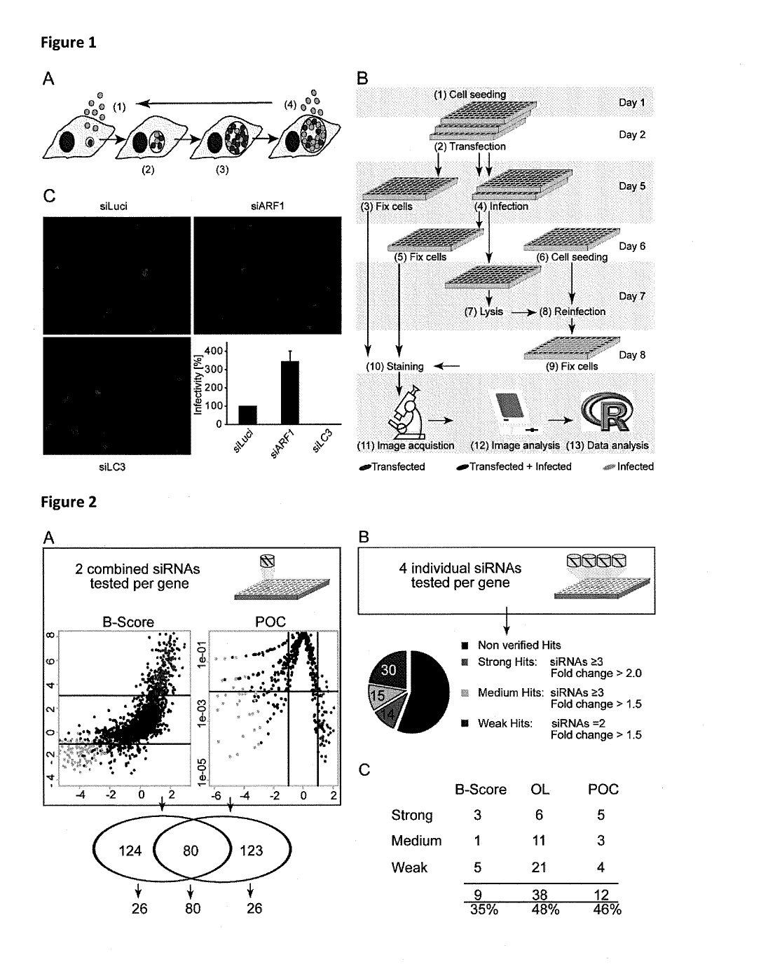

To identify host cell factors that might have crucial functions during Ctr infection and the progression of the pathogen's developmental cycle (FIG. 1A), we established a two-step assay that enabled us to determine (i) the number of EBs that infected cells or/and differentiated into RBs inside host cells (termed infection), or/and (ii) the resulting infectious progeny (termed infectivity). We used fluorescence microscopy as a read-out system (FIG. 1B). One day prior to transfection with small interfering RNAs (siRNAs), HeLa cells were seeded in three 96-well plates. The cells in one plate were fixed 72 hours post-transfection to exclude possible effects of gene knockdown on cell number. At the same time, cells in both of the remaining plates were infected with Ctr. Cells in one of the plates were used to monitor the infection rate 24 hours-post infection, whereas cells in the other plate were lysed 48 hours post-infection, and dilutions of the lysates were used to infect nontransfected HeLa cells, which were fixed 24 hours post-infection to monitor the infectivity rate of Ctr. All of the plates were then processed for immunofluorescence microscopy by staining the cell nuclei with Hoechst dye whereas bacterial inclusions were detected with an antibody against the major outer membrane protein (MOMP) of Ctr. The number of inclusions per cell and sizes of these inclusions were determined by automated microscopic readout.

To test the reliability of the functional assay, we used siRNAs specific for the small GTPase adenosine diphosphate (ADP)-ribosylation factor (ARF1) (siARF1), and a combination of siRNAs specific for the light-chain subunits of the microtubule-associated proteins MAP1 LC3A and MAP1 LC3B (siLC3). Transfection of cells with siARF1 prior to infection with Ctr resulted in larger inclusions and higher infectivity than occurred when cells were transfected with an siRNA (siLuci) against luciferase (thus, siARF1 was considered an activating control), whereas siLC3-mediated knockdown of MAP1 LC3A and MAP1 LC3B prior to infection resulted in the formation of smaller inclusions and almost no infectivity (FIG. 1C); thus, siLC3 was considered an inhibitory control Three siRNA libraries were screened: A kinase library that targeted 646 kinases and kinase-binding proteins, an apoptosis library directed against 418 apoptosis-related genes, and a custom library that targeted 471 genes with a broad range of cellular functions. Altogether, 1,289 unique genes were targeted with two pooled siRNAs per gene. Each pooled siRNA was tested a minimum of three times in 96-well plates. Only plates in which the controls showed increased or decreased infectivity rates of at least two-fold were analyzed further.

For quality control, a plate-wise correlation coefficient matrix was generated for each of the tested parameters in the assay, based on all samples. Data were normalized by B-Score and percent-of-control (POC) analyses (FIG. 2A), and targeted genes were designated as primary hits according to defined thresholds, as described in the Materials and Methods. With this approach, we identified 204 and 203 primary hits from the B-Score and POC analyses, respectively. For further analyses, we focused on the 80 genes common to both methods, in addition to 26 genes that were identified exclusively from either the B Score or POC methods, giving a total of 132 primary hits.

### 1b) Hit Validation

To validate the initial 132 hits, we performed a second round of screening that used four independent, newly designed siRNAs for each target gene (FIG. 2B). Data were normalized by POC. Validated hits that showed a minimum change in infectivity of two-fold with at least three siRNAs were classified as strong, those that exhibited a 1.5-fold effect with at least three siRNAs were classified as medium hits, and those that exhibited a 1.5-fold effect with two siRNAs were categorized as weak hits. Primary hits that did not meet the validation criteria or that showed opposing phenotypes were grouped as “not validated.” With these stringent criteria, of the 132 primary hits subjected to hit validation, 30 qualified as weak, 15 as medium, and 14 as strong hits (FIG. 2B, Table 1a). Of the primary hits that were exclusively derived from the B-Score and POC methods, we achieved a validation rate of 35% and 46%, respectively; a validation rate of 48% was achieved by combining both methods (FIG. 2C). These validation rates indicate that control-based normalization of RNAi screening data may be more reliable than sample-based normalization.

### 2) Importance of K-Ras and Raf-1

2a) Knockdown of K-Ras and Raf-1 leads to increased Ctr infectivity

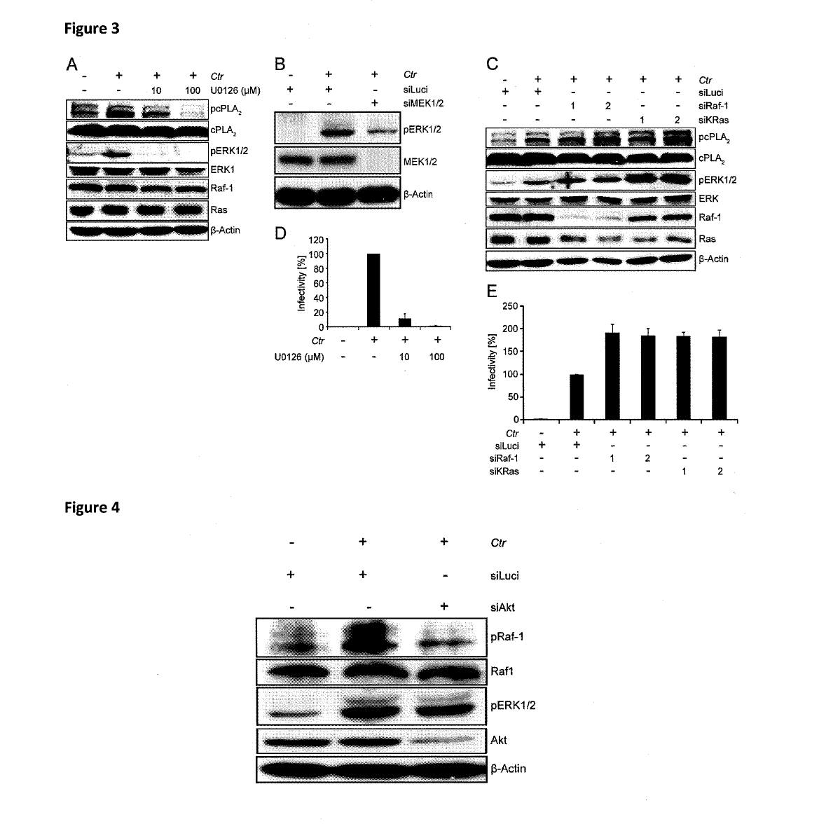

The Ras-Raf-MEK-ERK pathway is activated after infection with Ctr, which leads to the phosphorylation and activation of cPLA₂ by ERK (Su et al., 2004). Blocking the Ras-Raf-MEK-ERK pathway with chemical inhibitors, for example the MEK inhibitor U0126, decreases the infectivity of Ctr and reduces the extent of phosphorylation of cPLA₂ (Su et al., 2004). In contrast, our screening results showed that knockdown of K-Ras and Raf-1 led to increased Ctr infectivity (Table 1a). Knockdown of the other Raf and Ras family members failed to elicit equivalent increases in Ctr infectivity. To further elucidate the mechanism of by which the Ras-Raf-MEK-ERK pathway was activated during Ctr infection, we compared the cellular outcomes generated by chemical inhibitors with those caused by siRNA-mediated knockdown of gene expression. Western blotting analysis revealed that ERK and cPLA₂ were strongly phosphorylated 30 hours post-infection, whereas the MEK inhibitor U0126 repressed the phosphorylation of ERK and cPLA₂ in response to infection (FIG. 3A). Knockdown of MEK also hampered the phosphorylation of ERK after infection with Ctr (FIG. 3B), whereas ERK and cPLA₂ were still phosphorylated when K-Ras and Raf-1 were knocked down (FIG. 3C). Consistently, U0126 decreased the infectivity of Ctr (FIG. 3D), whereas knockdown of K-Ras and Raf-1 led to increased infectivity (FIG. 3E). These data strongly suggest that the phosphorylation of ERK and the phosphorylation and activation of cPLA₂ during Ctr infection require MEK but not K-Ras or Raf-1. In addition, both the activation of ERK and the depletion of K-Ras and Raf-1 supported the growth of Chlamydia within host cells. Thus, we further investigated the fate of Raf-1 during Ctr infection.

2B) Raf-1 is Phosphorylated at Ser²⁵⁹ after Ctr Infection

Because knockdown of Raf-1 supported the growth of Chlamydia, we investigated whether the phosphorylation of Raf-1 was influenced by Ctr infection. Previous studies showed that Raf-1 is inactivated when it is phosphorylated at Ser²⁵⁹ by Akt (Wu et al., 2008; Zimmermann and Moelling, 1999). Our Western blotting analysis revealed the increased abundance of Raf-1 phosphorylated at Ser²⁵⁹ in Ctr-infected cells compared to that in uninfected cells, and that knockdown of Akt inhibited this infection-dependent phosphorylation event (FIG. 4). These findings strongly suggested that Raf-1 was inactivated by Akt-dependent phosphorylation at Ser²⁵⁹ in response to infection by Ctr. Thus, by inhibition of Akt1, Akt2 or/and Akt, a chlamydial infection can be successfully treated. Akt1, Akt2 or/and Akt are suitable targets for the prophylaxis or/and treatment of an infection with a microorganism selected from the family Chlamydiaceae. Akt1, Akt2 or/and Akt may be used in a screening method, as described herein, for compounds suitable for the prophylaxis or/and treatment of an infection with a microorganism selected from the family Chlamydiaceae. Furthermore, an inhibitor of Akt1, Akt2 or/and Akt may be used for the prophylaxis or/and treatment of an infection with a microorganism selected from the family Chlamydiaceae.

Preferably, inhibition of Akt1, Akt2 or/and Akt includes inhibition of the interaction of Akt1, Akt2 or/and Akt with Raf-1.

2c) Phosphorylated Raf-1 is Recruited to the Inclusion in an Akt- and 14-3-3β-dependent manner

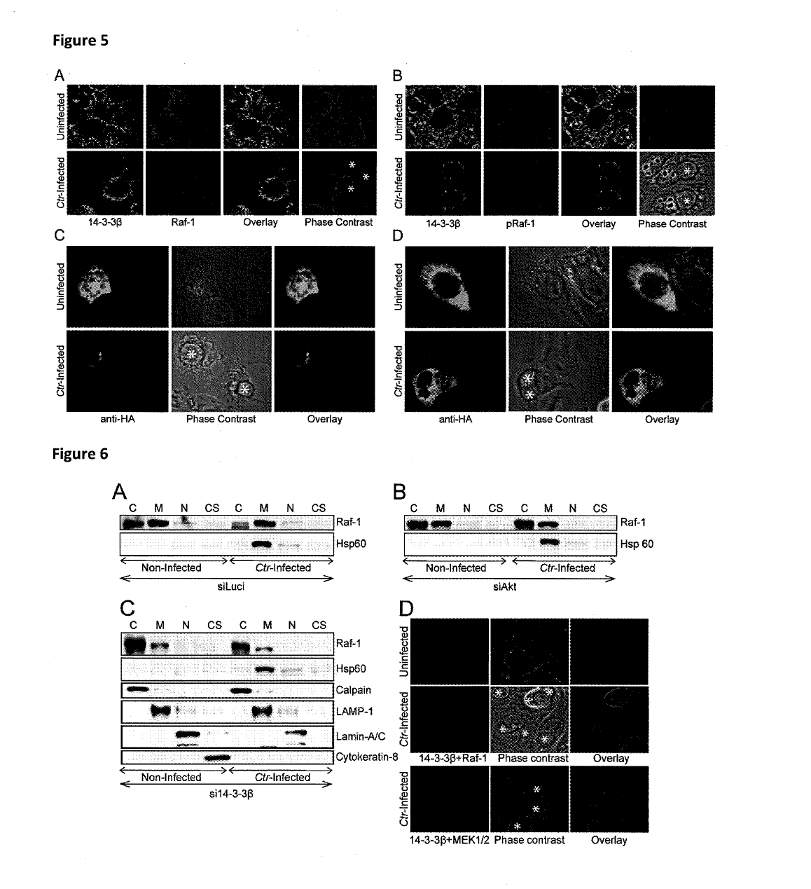

During Ctr infection, 14-3-3β is recruited to the inclusion by Inclusion protein G (IncG) (Scidmore and Hackstadt, 2001) and interacts with other host cell proteins, such as BAD (Verbeke et al., 2006). Phosphorylation of Raf-1 at Ser²⁵⁹ results in the binding of Raf-1 to 14-3-3β, a negative regulator of Raf-1 (Zimmermann and Moelling, 1999), and Raf-1 is redistributed within Chlamydia-infected cells (Chu et al., 2008). Thus, we speculated that Raf-1 might also be recruited to the inclusion upon infection in a 14-3-3β- and Akt-dependent manner. Uninfected and Ctr-infected HeLa cells were fixed 30 hours post-infection and incubated with antibodies against 14-3-3β, Raf-1, or phosphorylated Raf-1 (pRaf-1). Confocal images revealed that Raf-1 and pRaf-1 colocalized with 14-3-3β at the membranes of inclusions in infected cells, whereas in uninfected cells, Raf-1 and pRaf-1 were dispersed throughout the cytoplasm (FIG. 5, A and B). Additionally, ectopic expression of wild-type Raf-1 or a Ser²⁵⁹→Ala mutant of Raf-1 (S259A) revealed that only the wild-type protein localized to the inclusions, whereas the mutant form remained in the cytoplasm of infected cells (FIG. 5, C and D). These data confirmed the phosphorylation-dependent recruitment of Raf-1 to the inclusion.

To corroborate these observations, we performed fractionation experiments. Uninfected and Ctr-infected cells transfected with siRNAs specific for luciferase (a negative control), Akt, or 14-3-3β were lysed 30 hours post-infection, separated into subcellular fractions, and subjected to Western blotting analysis to detect Raf-1 and chlamydial heat shock protein 60 kD (Hsp60), as a marker for Chlamydia. As expected, chlamydial Hsp60 was found mainly in the membrane- and organelle-containing fraction of infected cells (FIG. 6, A to C). Consistent with our confocal results, Raf-1 was distributed between the cytosolic and the membrane- and organelle-containing fractions in uninfected, control cells transfected with an siRNA against luciferase. In contrast, Raf-1 was predominantly localized to the membrane- and organelle-containing fraction in infected cells (FIG. 6A). However, in Akt-knockdown cells, we observed a strong increase in the abundance of Raf-1 in the cytosolic fractions of both uninfected and Ctr-infected cells (FIG. 6B). A similar scenario was observed when cells were depleted of 14-3-3β (FIG. 6C). To investigate whether Raf-1 directly interacted with 14-3-3β at the inclusion, an in situ proximity ligation assay was performed, which enabled us to visualize protein-protein interactions. In Ctr-infected cells we clearly observed a strong accumulation of signals at the inclusion (FIG. 6D). Thus, our findings demonstrate pRaf-1 was recruited to the inclusion in a manner that was dependent on Akt and a direct interaction with 14-3-3β. Thus, by inhibition of 14-3-3β, a chlamydial infection can be successfully treated. 14-3-3β is a suitable target for the prophylaxis or/and treatment of an infection with a microorganism selected from the family Chlamydiaceae. 14-3-3β may be used in a screening method, as described herein, for compounds suitable for the prophylaxis or/and treatment of an infection with a microorganism selected from the family Chlamydiaceae. Furthermore, an inhibitor of 14-3-3β may be used for the prophylaxis or/and treatment of an infection with a microorganism selected from the family Chlamydiaceae.

Preferably, inhibition of 14-3-3β includes inhibition of the interaction of 14-3-3β with Raf-1, in particular phosphorylated Raf-1.

### 3) Summary

Here, we present an siRNA-based, loss-of-function screen in human epithelial cells that identified 59 targets that positively or negatively regulated C. trachomatis infectivity. Network and gene-enrichment analyses pointed towards K-Ras and Raf-1 as central players involved in several signaling networks engaged during Ctr infection. To validate this observation, we dissected the functions of K-Ras and Raf-1 during infection. We found that ERK was activated even when Raf-1 was depleted; that Raf-1 was phosphorylated at Ser', a known inactivating modification of Raf-1, in an Akt-dependent manner; and that phosphorylated Raf-1 was recruited to the inclusion, in a manner that was dependent on Akt and a direct interaction with 14-3-3β. These findings have revealed an unexpected Ras- and Raf-independent MEK-ERK signaling pathway during Ctr infection.

In conclusion, this is the first comprehensive, human cell-based, RNAi loss-of-function screen for host cell factors that either positively or negatively affect the developmental cycle of Ctr. Detailed investigation of two of these factors, Ras and Raf-1, demonstrated an uncoupled regulation of components of the canonical Ras-Raf-MEK-ERK signaling cascade by Chlamydia. Our study also provides evidence for the inactivation of Raf-1 during Ctr infection. The functional importance of this inactivation is currently under investigation; however, we hypothesize that Ctr specifically inactivates and sequesters Raf-1 to actively interfere with the downstream signaling events induced by Raf-1 independently of MEK and ERK. Our observations indicate that Ctr has evolved efficient strategies to uncouple individual modules from otherwise coherent signaling cascades and further advance our understanding of Chlamydia-host cell interactions.

### Materials and Methods

### Cell Lines and Bacterial Strains

HeLa cells (ATCC CCL-2) were grown in Hepes-buffered growth medium [RPMI (GibCo) supplemented with 10% fetal calf serum (FCS) (Biochrome), 2 mM glutamine, and 1 mM sodium pyruvate], at 37° C. in a humidified incubator containing 5% CO₂. Ctr serovar L2 (ATCC VR-902B) was propagated in HeLa cells in infection medium (RPMI medium supplemented with 5% FCS).

### Propagation of Chlamydia and Infections

Ctr was propagated in HeLa cells grown in 150-cm² cell culture flasks in 24 ml of infection medium. The cells were detached 48 hours after infection with 3-mm glass beads and were centrifuged at 500 g, for 10 min at 4° C. The pelleted cells were resuspended in sucrose-phosphate-glutamate (SPG) buffer and ruptured by vortexing with glass beads. Cell lysates were then centrifuged as before to sediment nuclei and cell debris. The supernatant was further centrifuged at 20,000 g for 40 min at 4° C. and the resulting bacterial pellet was resuspended in 15 ml of SPG buffer with a 21- to 22-gauge injection needle. Suspensions of Chlamydia were stored in aliquots at −75° C. until required. HeLa cells were infected with Ctr at a multiplicity of infection (MOI) of 0.5 to 3 in infection medium. The medium was refreshed 2 hours p.i, and the cells were grown at 35° C. in 5% CO₂ until they were fixed or used lysed to be used for reinfections.

Transfection of Cells with siRNAs

All siRNAs were purchased from Qiagen. The siRNAs of the custom library were validated at the Max Planck Institute for Infection Biology, Berlin, for their ability to knockdown mRNA expression of target genes by more than 70% compared to control cells transfected with siRNA specific for luciferase, as described previously (Machuy et al., 2005). Transfection of cells in 96-well plates with siRNAs was performed with the BioRobot 8000 system (Qiagen). One day prior to transfection, 1.5×10³ HeLa cells were seeded in each well of a 96-well plate. For each well, 5 μl of the siRNA stock solution (0.2 μM) was resuspended in 15 μl of RPMI without serum and incubated at room temperature for 10 min, to which was added 10 μl of a 1:20 diluted solution of Hiperfect (Qiagen) and the mixture was incubated at room temperature for a further 10 min before 25 ml of growth medium was added. 50 μl of this transfection mixture was added to each well of the plate in addition to 50 μl of growth medium, which resulted in a final concentration of siRNA of 10 nM. Cells were incubated at 37° C. and 5% CO₂ for 72 hours. For the analysis of functional experiments by Western blotting, 1×10⁵ cells were seeded into each well of a 12-well plate 24 hours prior to transfection. Cells were then transfected with Hiperfect transfection reagent according to the manufacturer's guidelines. In brief, 150 ng of specific siRNA was added to RPMI without serum and incubated with 6 μl of Hiperfect in a total volume of 100 μl. After 10 to 15 min, the liposome-siRNA mixture was added to the cells with 1 ml of cell culture medium, which gave a final concentration of siRNA of 10 nM. After 1 day, cells were trypsinized and seeded into new cell culture plates, depending on the experiments. Three days post-transfection, the cells were infected and incubated as indicated above.

### Infectivity Assays

In 96-well plates, HeLa cells were infected as described above. At 2 days post-infection, with a BioRobot 8000 system, cells were lysed by adding Nonidet P40 (NP40) (Fluka) at a final concentration of 0.06% for 15 min at room temperature. HeLa cells in 6-well plates were infected with Ctr for 48 hours and then were scraped off the plates with a rubber policeman. The cells were collected in 15-ml tubes containing sterile glass beads and lysed by vortexing (at 2,500 rpm for 3 min). For both plate formats, lysates were then diluted 1:100 in infection medium before being transferred to fresh, untreated HeLa cells. After incubation at 35° C. and 5% CO₂ for 24 hours, the cells were fixed in ice-cold methanol overnight at 4° C. and then processed with the indirect immunofluorescence protocol described below.

### Antibodies

Antibodies were obtained from the following sources: Rabbit antibodies against Raf-1, Ras, phosphorylated cPLA₂, total cPLA₂, total p44 MAPK (ERK1), phosphorylated Raf-1 at Ser²⁵⁹, LAMP-1, MEK1 and MEK2, Akt, calpain and mouse antibodies against phosphorylated p44 and p42 MAPK (ERK1 and ERK2) were purchased from Cell Signaling Technology. Goat and mouse antibodies against 14-3-3β and rabbit antibodies against Raf-1 (H-71), cytokeratin-8, and the HA eptiope (Y-11) were purchased from Santa Cruz Biotechnology. Mouse antibody against lamin-A/C was obtained from Chemicon, mouse antibody against Chlamydia Hsp60 was purchased from Alexa, mouse antibody against β-actin was from Sigma, and mouse antibody against Chlamydia MOMP KK12 was from the University of Washington. Secondary antibodies conjugated to horseradish peroxidase (HRP) were purchased from Amersham Biosciences and secondary antibodies labeled with the fluorochromes Cy2, Cy3, and Cy5 were from Jackson Immuno Research Laboratories.

### Indirect Immunofluorescence Labeling

Fixed cells (in 96-well and 6-well plates) were washed twice with phosphate-buffered saline (PBS) and blocked by incubating with 0.2% bovine serum albumin (BSA) in PBS (blocking buffer) for 30 min at room temperature. Primary mouse antibody against C. trachomatis MOMP KK12 (at a 1 in 10,000 dilution) was added to the cells for 1 hour at room temperature before washing twice with PBS. The Cy3-labeled goat antibody against mouse immunoglobulin G (IgG) was then added at a 1 in 100 dilution for 1 hour. Host cell nuclei were stained with Hoechst 33342 (Sigma) at a 1 in 2,000 dilution.

Double Labeling of Raf-1 or pRaf-1 and 14-3-3β and Confocal Microscopy

Infected cells were grown on coverslips, washed twice with PBS, and then fixed with ice-cold methanol overnight at 4° C. Cells were washed again with PBS two times and then incubated in blocking buffer as described earlier. The cells were then incubated for 1 hour at room temperature with antibody against 14 3-3β together with antibody against Raf-1 or pRaf-1 (Ser²⁵⁹) in 100 μl of blocking buffer. The cells were then incubated for 1 hour at room temperature with the appropriate fluorochrome-conjugated secondary antibodies at a 1 in 100 dilution. Between incubation steps, cells were washed with PBS three times. Coverslips were washed and mounted on glass microscopic slides with Moviol. The fluorochromes were visualized with Cy2 and Cy5 filters. A series of images with Z stacks were acquired with a laser scanning confocal microscope (Leica) and analyzed with Imaris Software (Bitplane) and further processed with Photoshop CS3 (Adobe Systems).

Treatment of Cells with U0126

Cells (1×10⁵) were seeded in each well of a 12-well plate one day prior to infection. Two hours after infection with Ctr (at an MOI of 3), 1 ml of fresh infection medium containing either 10 μM or 100 μM U0126 was added to the cells. Depending on the experiment cells were harvested for western blotting analysis or for determination of infectivity.

### Automated Microscopy and Image Analysis

The numbers and sizes of chlamydial inclusions and host cells were analyzed with an automated microscope (Olympus Soft Imaging Solutions). Images were taken with DAPI and Cy3 filtersets (AHF-Analysetechnik) at the same position. ScanR Analysis Software (Olympus Soft Imaging Solutions) was used to automatically identify and quantify inclusions and cells.

### Subcellular Fractionations

Subcellular fractionation was carried out with the ProteoExtract Subcellular Proteome Extraction kit (Calbiochem), according to the manufacturer's instructions.

Transfections with pcDNA3

HeLa cells were grown on coverslips in 12-well plates, transfected with 1 μg of plasmid DNA encoding HA-tagged WT Raf-1 (pcDNA3-Raf-1-WT) or the HA-tagged S259A mutant of Raf-1 (pcDNA3-Raf-1-S259A) with Lipofectamine 2000 (Invitrogen), as described by the manufacturer. Twenty-four hours later, cells were infected with Ctr at an MOI of 2. Thirty hours post-infection, cells were washed twice with PBS and fixed with ice-cold methanol overnight at 4° C. Cells were washed again in PBS two times and then incubated with blocking buffer as described earlier. The cells were then incubated with primary antibody against the HA tag for 1 hour at room temperature. Cells were then incubated with the secondary fluorochrome-conjugated antibody at a 1 in 100 dilution for 1 hour at room temperature. Between incubation steps, cells were washed with PBS three times. Coverslips were washed and mounted on glass microscopic slides with Moviol. Images were acquired with a fluorescent microscope (Leica) and processed with Photoshop CS3 (Adobe Systems).

### Proximity Ligation Assay

HeLa cells grown on coverslips in 12-well plates, were infected with Ctr, 30 h post-infection washed twice with PBS 30 hours post-infection, and then fixed with ice-cold methanol overnight at 4° C. Incubation with antibodies against Raf-1 (H-71), or MEK1/2, or 14-3-3β (A-6) was performed with the Proximity Ligation Assay kit (OLINK) according to the manufacturer's instructions. A series of images with Z stacks were acquired with a laser scanning confocal microscope (Leica) and analyzed with Imaris Software (Bitplane) and further processed by Photoshop CS3 (Adobe Systems).

### SDS-PAGE and Western Blotting

Depending on the experiment, untransfected or transfected HeLa cells were grown in six-well plates, infected with Ctr as described earlier, and then washed with PBS. To each well was added 200 μl of 1×SDS sample buffer (3% 2-mercaptoenthanol, 20% glycerin, 0.05% bromphenol blue, 3% SDS). Cell lysates were collected and boiled for 10 min. Samples were stored at −20° C. until required. Proteins from the cell lysates were resolved by SDS-PAGE, transferred to polyvinylidene difluoride (PVDF) membranes (PerkinElmer Life Sciences) and blocked with 3% milk powder in Tris-buffered saline (containing 0.5% Tween 20) for 30 min before incubation with the appropriate antibodies. The bound primary antibodies were incubated with the corresponding HRP-conjugated secondary antibodies. Immunoreactive proteins were detected on an X-ray film directly or with the AIDA Image Analyzer after addition of ECL reagent (Amersham Biosciences).

### Statistical Analysis

Screening data were corrected for plate-to-plate variability by normalizing compound measurements relative to controls with POC and B-score analyses (Malo et al., 2006). The resulting data from both methods were used for further analysis and hit classification. For the POC method, P values and log₂ ratios were calculated for each of the samples. Hits were then classified by defining P value (<0.05) and fold change (>2) for the primary screen, and fold change (>1.5) for the hit validation. In the B-Score method, hits were scored by transforming the normalized measurements into Z-scores. Hits were then classified by defining thresholds of the Z-score for both up-regulating and down-regulating phenotypes (3 and −1, respectively).

### Gene Enrichment and Network Analysis

For gene enrichment analysis, we modified the R-script available from the Gaggle website at the following URL: http://gaggle.systemsbiology.net/svn/gaggle/PIPE2.0/trunk/PIPEletResource Dir/GOTableEnrichment/GOEnrichmentScript.R. This script applies the R-package GOstats developed by Falcon and Gentleman (Falcon and Gentleman, 2007) and is available at Bioconductor (http://www.bioconductor.org). Briefly, we defined a gene universe consisting of 1,289 genes targeted in our screen and processed different gene hit lists (strong, medium, and weak) against this universe with respect to molecular function (MF), cellular component (CC), and biological process (BP). For the significantly enriched gene ontology terms, we calculated the enrichment factors. Network analysis was carried out with Ingenuity Pathway Analysis (IPA) software (http://www.ingenuity.com/).

### EXAMPLE 2

### Identification of Target Genes in Host Cell Nucleotide Metabolism that are Essential for Chlamydia Growth, Propagation or/and Infection

We performed a genome-wide siRNA-based screen in human epithelial cells to identify host cell factors that are essential for Chlamydia infection using the Qiagen Hu_Genome Set V1.0 and the Human Druggable Genome siRNA Set V2.0. C. trachomatis L2 was used. In the primary screen we identified 60 sequences that target genes involved in nucleotide metabolism and that showed a strong inhibitory effect on the formation of Chlamydia infectious progeny. The results are summarized in Table 1b.

### EXAMPLE 3

Our experiments using RNAi have shown that knockdown of human GMP synthase (GMPS) inhibits the intracellular replication of Chlamydia and the formation of infectious progeny.

Thus, by inhibition of GMPS, a chlamydial infection can be successfully treated. GMPS is a suitable target for the prophylaxis or/and treatment of an infection with a microorganism selected from the family Chlamydiaceae. GMPS may be used in a screening method, as described herein, for compounds suitable for the prophylaxis or/and treatment of an infection with a microorganism selected from the family Chlamydiaceae. Furthermore, an inhibitor of GMPS may be used for the prophylaxis or/and treatment of an infection with a microorganism selected from the family Chlamydiaceae.

The involvement of GMPS in Chlamydia infection has not been demonstrated so far. According to the state of the art, GMPS is required for the novo GMP synthesis but is also involved in transcriptional control, at least in part, through cooperation with USP7.

GMP synthase (GMPS, E.C. 6.3.5.2) is a glutamine amidotransferase involved in the de novo synthesis of purines. It catalyzes the conversion of xanthosine 5′-monophosphate to guanosine 5′-monophosphate in the presence of glutamine and ATP. GMPS is a bifunctional enzyme with two domains, an N-terminal glutaminase domain that generates ammonia from glutamine, and a C-terminal synthethase domain that aminates XMP to form GMP (Hirst et al., 1994, Nakamura et al., 1995).

It has been shown that GMPS has increased activity in highly proliferating cells and thus, it is a potential target for anticancer therapies. Glutamine analogs, like acivicin have been shown to inhibit GMPS (Chittur et al., 2001).

In Drosophila embryos GMPS is tightly associated with the ubiquitin-specific protease 7 (USP7) and contributes to epigenetic silencing of homeotic genes by Polycomb. The USP7-GMPS complex catalyzes the selective deubiquitylation of histone H2B. Indeed, USP7 binding to GMPS strongly augmented deubiquitylation of the human tumor suppressor p53 (Van der Knaap et al., 2005). Further, the GMPS-USP7 complex binds and regulates ecdysone target loci, implicating a complex of a biosynthetic enzyme and ubiquitin protease in gene control by hormone receptors (Van der Knaap et al., 2010).

Sarkari et al. (2009), has shown an interaction of USP7 with GMPS in human cells. After Epstein—Barr virus (EBV) infection, this interaction stimulates the ability of USP7 to cleave monoubiquitin from histone H2B. Here, the USP7-GMPS complex forms a quaternary complex with DNA-bound EBNA1 enabling the persistence of EBV genomes in infected cells.

The effect of chemical inhibitors of GMPS on Chlamydia infection was tested. Using acivicin we observed a complete block in Chlamydia replication. Decoyinine, an analogue of adenosine, which is used to block GMPS (Zhang et al., 2005), showed no inhibitory effect on Chlamydia replication.

We were able to recover Chlamydia replication by addition of the nucleotides GTP and GMP to GMPS knockdown cells and to cells to which acivicin was added, demonstrating GMPS to be essential for Chlamydia growth through its function in GMP synthesis.

In immunofluorescence staining studies we observed recruitment of GMPS to the Chlamydia inclusion. We applied the Proximity ligation Assay (PLA from OLink) to investigate a possible interaction of GMPS with HAUSP in Chlamydia infection and find interaction of GMPS and HAUSP.

### REFERENCES

Brunham R C, Rey-Ladino J (2005). Immunology of Chlamydia infection: implications for a Chlamydia trachomatis vaccine. Nat Rev Immunol 5: 149-61.
Chu H G, Weeks S K, Gilligan D M, Rockey D D (2008). Host alpha-adducin is redistributed and localized to the inclusion membrane in chlamydia- and chlamydophila-infected cells. Microbiology 154: 3848-55.
Falcon S, Gentleman R (2007). Using GOstats to test gene lists for GO term association. Bioinformatics 23: 257-8.
Machuy N, Thiede B, Rajalingam K, Dimmler C, Thieck O, Meyer T F et al (2005). A global approach combining proteome analysis and phenotypic screening with RNA interference yields novel apoptosis regulators. Mol Cell Proteomics 4: 44-55.
Malo N, Hanley J A, Cerquozzi S, Pelletier J, Nadon R (2006). Statistical practice in high-throughput screening data analysis. Nat Biotechnol 24: 167-75.
Moulder J W (1991). Interaction of chlamydiae and host cells in vitro. Microbiol. Rev 55: 143-90.
Rommel C, Radziwill G, Lovric J, Noeldeke J, Heinicke T, Jones D et al (1996). Activated Ras displaces 14-3-3 protein from the amino terminus of c-Raf-1. Oncogene 12: 609-19.
Scidmore M A, Hackstadt T (2001). Mammalian 14-3-3beta associates with the Chlamydia trachomatis inclusion membrane via its interaction with IncG. Mol Microbiol 39: 1638-50.
Su H, McClarty G, Dong F, Hatch G M, Pan Z K, Zhong G (2004). Activation of Raf/MEK/ERK/cPLA2 signaling pathway is essential for chlamydial acquisition of host glycerophospholipids. J Biol Chem 279: 9409-16.
Verbeke P, Welter-Stahl L, Ying S, Hansen J, Hacker G, Darville T et al (2006). Recruitment of BAD by the Chlamydia trachomatis vacuole correlates with host-cell survival. PLoS Pathog 2: e45.
Wright H R, Turner A, Taylor H R (2008). Trachoma. Lancet 371: 1945-54.
Wu H W, Li H F, Wu X Y, Zhao J, Guo J (2008). Reactive oxygen species mediate ERK activation through different Raf-1-dependent signaling pathways following cerebral ischemia. Neurosci Lett 432: 83-7.
Zimmermann S, Moelling K (1999). Phosphorylation and regulation of Raf by Akt (protein kinase B). Science 286: 1741-4.
Hirst M, Haliday E, Nakamura J, Lou L. Human GMP synthetase. Protein purification, cloning, and functional expression of cDNA. J Biol. Chem. 1994 Sep. 23; 269(38):23830-7.
Nakamura J, Straub K, Wu J, Lou L. The glutamine hydrolysis function of human GMP synthetase. Identification of an essential active site cysteine. J Biol. Chem. 1995 Oct. 6; 270(40):23450-5.
Chittur S V, Klem T J, Shafer C M, Davisson V J. Mechanism for acivicin inactivation of triad glutamine amidotransferases. Biochemistry. 2001 Jan. 30; 40(4):876-87.
Van der Knaap, W A., Kumar, B. R., Moshkin, Y., Langenberg, K., Krijgsveld, J., Heck, A., Karch, F., and Verrijzer, P. GMP synthethase stimulates histone H2B deubiquitylation by the epigenetic silencer USP7. Molecular Cell, 2005; 17:695-707.
Van der Knaap, J. A., Kozhevnikova, E., Langenberg, K., Moshkin, Y., and Verrijzer, P. Biosynthetic enzyme GMP synthethase cooperates with ubiquitin-specific protease 7 in transcriptional regulation of ecdysteroid target genes. Molecular and Cellular Biology, 2010; 30(3):736-744.
Sarkara, F., Sanchez-Alcaraz, T., Wang, S., holoway, M., Sheng, Y. and Frappier, L. PLOS pathogens. EBNA1-mediated recruitment of a histone H2B deubiquitylating complex of the Epstein-Barr virus latent origin of DNA replication. 2009; 5(10):1-14.
Zhang S, Haldenwang W G. Contributions of ATP, GTP, and redox state to nutritional stress activation of the Bacillus subtilis sigmaB transcription factor. J. Bacteriol. 2005 November; 187(22):7554-60.
Peter J. O'Dwyer, M. Teresa Alonso, and Brian Leyland-Jones. Acivicin: A New Glutamine Antagonist in Clinical Trials. Journal of Clinical Oncology, 1984; Vol 2, No 9 (September).
Weber G, Nagai M, Natsumeda Y, Ichikawa S, Nakamura H, Eble J N, Jayaram H N, Zhen W N, Paulik E, Hoffman R, et al. Regulation of de novo and salvage pathways in chemotherapy. Adv Enzyme Regul. 1991; 31:45-67.
G. L. Neil, A E. Berger, R P. McPartland, G. B. Grindey and A. Bloch. Biochemical and pharmacological effects of the fermentation-derived antitumor agent, (aS,5S)-a-ammo-3-chloro-4,5-dihydro-5-1soxazoleacetac acid (AT-125). Cancer Res. 1979; 39, 852-856.
Mukherjee T, Roy K, Bhaduri A. Acivicin: a highly active potential chemotherapeutic agent against visceral leishmaniasis. Biochem Biophys Res Commun. 1990 Jul. 31; 170(2):426-32.
Vilmont M, Azoulay M, Frappier F. Metabolism of glutamine in erythrocytes infected with the human malaria parasite: Plasmodium falciparum. Ann Parasitol Hum Comp. 1990; 65(4):162-6.
A. Hofer, D. Steverding, A. Chabers, R. Brun, L. Thelander, Proc. Natl. Acad. Sci. USA 2001, 98, 6412-6416.
A. Fijolek, A. Hofer, L. Thelander, J. Biol. Chem. 2007, 282-282, 11 858-11 865.
Orth R, Böttcher T, Sieber S A. The biological targets of Acivicin inspired 3-chloro- and 3-bromodihydroisoxazole scaffolds. Chem Commun (Camb). 2010 Nov. 28; 46(44):8475-7.

TABLE 1
Table 1a

| | | | | | Infectivity |
|---|---|---|---|---|---|
| NCBI | Entrez | | | | phenotype |
| gene | Gene | | mRNA | sIRNA | upon |
| symbol | Id | Gene Description | Accessions | Target Sequence | Knockdown |
| PTEN | 5728 | phosphatase and tensin homolog | NM_000314 | ACGGGAAGACAAGTTCATGTA | Down |
| | | (mutated in multiple advanced cancers 1) | | | |
| PTEN | 5728 | phosphatase and tensin homolog | NM_000314 | TCGGCTTCTCCTGAAAGGGAA | Down |
| | | (mutated in multiple advanced cancers 1) | | | |
| PTEN | 5728 | phosphatase and tensin homolog | NM_000314 | ATCGATAGCATTTGCAGTATA | Down |
| | | (mutated in multiple advanced cancers 1) | | | |
| COPB1 | 1315 | coatomer protein complex, subunit beta 1 | NM_016451 | CAGGATCACACTATCAAGAAA | Down |
| COPB1 | 1315 | coatomer protein complex, subunit beta 1 | NM_016451 | CAGAATTGCTAGAACCTTTAA | Down |
| COPB1 | 1315 | coatomer protein complex, subunit beta 1 | NM_016451 | CACCAACATGGTTGATTTAAA | Down |
| CDKN1C | 1028 | cyclin-dependent kinase inhibitor 1C (p57, Kip2) | NM_000076 | CGCCAACGGCGCGGCGATCAA | Down |
| CDKN1C | 1028 | cyclin-dependent kinase inhibitor 1C (p57, Kip2) | NM_000076 | ACAGGCGAACCCGACGCAGAA | Down |
| CDKNIC | 1028 | cyclin-dependent kinase inhibitor 1C (p57, Kip2) | NM_000076 | TCGGCTGGGACCGTTCATGTA | Down |
| FGFR3 | 2261 | fibroblast growth factor receptor 3 | NM_000142 | AAGGTTTATCCCGCCGATAGA | Down |
| | | (achondroplasia, thanatophoric dwarfism) | NM_022965 | | |
| FGFR3 | 2261 | fibroblast growth factor receptor 3 | NM_000142 | CAGGAGAATTAGATTTCTATA | Down |
| | | (achondroplasia, thanatophoric dwarfism) | NM_022965 | | |
| FGFR3 | 2261 | fibroblast growth factor receptor 3 | NM_000142 | CTGGACCTGTATATTTGTAAA | Down |
| | | (achondroplasia, thanatophoric dwarfism) | NM_022965 | | |
| MAPK14 | 1432 | mitogen-activated protein kinase 14 | NM_001315 | CTCCGAGGTCTAAAGTATATA | Up |
| | | | NM_139012 | | |
| | | | NM_139013 | | |
| | | | NM_139014 | | |
| MAPK14 | 1432 | mitogen-activated protein kinase 14 | NM_001315 | CACGGGAACTCTCCAAATATT | Up |
| | | | NM_139012 | | |
| | | | NM_139013 | | |
| | | | NM_139014 | | |
| MAPK14 | 1432 | mitogen-activated protein kinase 14 | NM_001315 | AAGATGAACTTTGCGAATGTA | Up |
| | | | NM_139012 | | |
| | | | NM_139013 | | |
| | | | NM_139014 | | |
| DIABLO | 56616 | diablo homolog (Drosophila) | NM_019887 | GCGGTGTTTCTCAGAATTGAT | Up |
| | | | NM_138929 | | |
| DIABLO | 56616 | diablo homolog (Drosophila) | NM_019887 | CCAGAGCTGAGATGACTTCAA | Up |
| | | | NM_138929 | | |
| | | | NM_138930 | | |
| DIABLO | 56616 | diablo homolog (Drosophila) | NM_019887 | CTCCCGGAAAGCAGAAACCAA | Up |
| | | | NM_138929 | | |
| | | | NM_138930 | | |
| GUK1 | 2987 | guanylate kinase 1 | NM_000858 | CTGCGGCAGCGCAACACTGAA | Up |
| GUK1 | 2987 | guanylate kinase 1 | NM_000858 | CACCGATCTGCGGCCCATCTA | Up |
| GUK1 | 2987 | guanylate kinase 1 | NM_000858 | CACGAGCAAGGTGGCGGTGCA | Up |
| GRK5 | 2869 | G protein-coupled receptor kinase 5 | NM_005308 | CAGGAATAATGCGGTAGGCAA | Up |
| GRK5 | 2869 | G protein-coupled receptor kinase 5 | NM_005308 | CCCGCCAGATCTGAACAGAAA | Up |
| GRK5 | 2869 | G protein-coupled receptor kinase 5 | NM_005308 | CAGGTTCGGGCCACGGGTAAA | Up |
| RAF1 | 5894 | v-raf-1 murine leukemia viral oncogene homolog 1 | NM_002880 | GTGGATGTTGATGGTAGTACA | Up |
| RAF1 | 5894 | v-raf-1 murine leukemia viral oncogene homolog 1 | NM_002880 | CCCAGATCTTAGTAAGCTATA | Up |
| RAF1 | 5894 | v-raf-1 murine leukemia viral oncogene homolog 1 | NM_002880 | AACAGTGGTCAATGTGCGAAA | Up |
| TNIK | 23043 | TRAF2 and NCK interacting kinase | NM_015028 | CAGACGTCTCCCACTGATGAA | Up |
| TNIK | 23043 | TRAF2 and NCK interacting kinase | NM_015028 | CACCTATGGCCGGATAACTAA | Up |
| TNIK | 23043 | TRAF2 and NCK interacting kinase | NM_015028 | CCGGAATATTGCTACATACTA | Up |
| PTK2 | 5747 | PTK2 protein tyrosine kinase 2 | NM_005607 | AACAATTTATGTTCACATTAA | Up |
| | | | NM_153831 | | |
| PTK2 | 5747 | PTK2 protein tyrosine kinase 2 | NM_005607 | GAGCGTCTAATCCGACAGCAA | Up |
| | | | NM_153831 | | |
| PTK2 | 5747 | PTK2 protein tyrosine kinase 2 | NM_005607 | AAGTCTAACTATGAAGTATTA | Up |
| | | | NM_153831 | | |
| KRAS | 3845 | v-Ki-ras2 Kirsten rat sarcoma viral oncogene homolog | NM_004985 | GACGATACAGCTAATTCAGAA | Up |
| | | | NM_033360 | | |
| KRAS | 3845 | v-Ki-ras2 Kirsten rat sarcoma viral oncogene homolog | NM_004985 | GTGGACGAATATGATCCAACA | Up |
| | | | NM_033360 | | |
| KRAS | 3845 | v-Ki-ras2 Kirsten rat sarcoma viral oncogene homolog | NM_004985 | CAGACGTATATTGTATCATTT | Up |
| | | | NM_033360 | | |
| KRAS | 3845 | v-Ki-ras2 Kirsten rat sarcoma viral oncogene homolog | NM_004985 | AAGGAGAATTTAATAAAGATA | Up |
| | | | NM_033360 | | |
| TAXIBP1 | 8887 | Taxi (human T-cell leukemia virus type I) | NM_006024 | AGGGTGAAATTCGTGGAGCAA | Up |
| | | binding protein 1 | | | |
| TAXIBP1 | 8887 | Taxi (human T-cell leukemia virus type I) | NM_006024 | TTCAATGTTACTGCACTGAAA | Up |
| | | binding protein 1 | | | |
| TAXIBP1 | 8887 | Taxi (human T-cell leukemia virus type I) | NM_006024 | ACAGCACAACTTCGAGAACAA | Up |
| | | binding protein 1 | | | |
| TAXIBP1 | 8887 | Taxi (human T-cell leukemia virus type I) | NM_006024 | AAGGGTCTTACTGAAGTAACA | Up |
| | | binding protein 1 | | | |
| RHOA | 387 | ras homolog gene family, member A | NM_001664 | TACCCAGATACCGATGTTATA | Up |
| RHOA | 387 | ras homolog gene family, member A | NM_001664 | ATGATTATTAACGATGTCCAA | Up |
| RHOA | 387 | ras homolog gene family, member A | NM_001664 | TTCGGAATGATGAGCACACAA | Up |
| RHOA | 387 | ras homolog gene family, member A | NM_001664 | TACCTTATAGTTACTGTGTAA | Up |
| MAP3K9 | 4293 | mitogen-activated protein kinase kinase kinase 9 | NM_033141 | CACGACCATCTTTCACGAATA | Down |
| MAP3K9 | 4293 | mitogen-activated protein kinase kinase kinase 9 | NM_033141 | CTGGACGGCCGTGTTCGAGTA | Down |
| MAP3K9 | 4293 | mitogen-activated protein kinase kinase kinase 9 | NM_033141 | ACCATAGAGAATGTTCGCCAA | Down |
| MAP2K6 | 5608 | mitogen-activated protein kinase kinase 6 | NM_002758 | GGCCTTGGAATCTATAGTATA | Down |
| | | | NM_031988 | | |
| MAP2K6 | 5608 | mitogen-activated protein kinase kinase 6 | NM_002758 | TACGGTAGTGATGAAATTATA | Down |
| | | | NM_031988 | | |
| MAP2K6 | 5608 | mitogen-activated protein kinase kinase 6 | NM_002758 | CTCATCAATGCTCTCGGTCAA | Down |
| | | | NM_031988 | | |
| LITAF | 9516 | lipopolysaccharide-induced TNF factor | NM_004862 | ACCCTGAGTCCTGCCATCTAA | Down |
| LITAF | 9516 | lipopolysaccharide-induced TNF factor | NM_004862 | CAAGGTCGTAAATGCATGCTA | Down |
| LITAF | 9516 | lipopolysaccharide-induced TNF factor | NM_004862 | CTGGGCCTGAACATAATTTCA | Down |
| LITAF | 9516 | lipopolysaccharide-induced TNF factor | NM_004862 | CACCTCCATCCTATGAAGAGA | Down |
| WNK4 | 65266 | WNK lysine deficient protein kinase 4 | NM_032387 | CACTAGTGTCTCAGACCAGAA | Up |
| WNK4 | 65266 | WNK lysine deficient protein kinase 4 | NM_032387 | CAAGGTCACTTCGGGCAGAAA | Up |
| WNK4 | 65266 | WNK lysine deficient protein kinase 4 | NM_032387 | CTCGGGCACGCTCAAGACGTA | Up |
| WNK4 | 65266 | WNK lysine deficient protein kinase 4 | NM_032387 | AAGACGGTGTATCGAGGGCTA | Up |
| BUB1B | 701 | BUB1 budding uninhibited by benzimidazoles 1 homolog | NM_001211 | CAGGTAGACCTGTTTGGTATA | Up |
| | | beta (yeast) | | | |
| BUB1B | 701 | BUB1 budding uninhibited by benzimidazoles 1 homolog | NM_001211 | AAGCTCGAGTGTCTCGGCAAA | Up |
| | | beta (yeast) | | | |
| BUB1B | 701 | BUB1 budding uninhibited by benzimidazoles 1 homolog | NM_001211 | AAGGAGACAACTAAACTGCAA | Up |
| | | beta (yeast) | | | |
| DGKB | 1607 | diacylglycerol kinase, beta 90 kDa | NM_004080 | ATGGACCAATGCCAGGGTTAA | Up |
| | | | NM_145695 | | |
| DGKB | 1607 | diacylglycerol kinase, beta 90 kDa | NM_004080 | CTGCATTAAAGTGCTAATGAT | Up |
| | | | NM_145695 | | |
| DGKB | 1607 | diacylglycerol kinase, beta 90 kDa | NM_004080 | AGCAAGTGAGTTGCCCATTAA | Up |
| | | | NM_145695 | | |
| AATF | 26574 | apoptosis antagonizing transcription factor | NM_012138 | CACCTATTGACCATACTACAA | Up |
| AATF | 26574 | apoptosis antagonizing transcription factor | NM_012138 | TTGGTGCCTTTGAACGCTCAA | Up |
| AATF | 26574 | apoptosis antagonizing transcription factor | NM_012138 | CGGGAAGTGAGGAGATTTCTA | Up |
| MAPK12 | 6300 | mitogen-activated protein kinase 12 | NM_002969 | CGGCGCTAAGGTGGCCATCAA | Up |
| MAPK12 | 6300 | mitogen-activated protein kinase 12 | NM_002969 | CACAGGCAAGACGCTGTTCAA | Up |
| MAPK12 | 6300 | mitogen-activated protein kinase 12 | NM_002969 | CTGGGAGGTGCGCGCCGTGTA | Up |
| MAP3K14 | 9020 | mitogen-activated protein kinase kinase kinase 14 | NM_003954 | CAGGACTCACGTAGCATTAAA | Up |
| MAP3K14 | 9020 | mitogen-activated protein kinase kinase kinase 14 | NM_003954 | CCCGCTTGGATCAGTGACCAT | Up |
| MAP3K14 | 9020 | mitogen-activated protein kinase kinase kinase 14 | NM_003954 | CCCGTGTGTGTTGGAAGGGAA | Up |
| TAOK3 | 51347 | TAO kinase 3 | NM_016281 | ACGCAGCGAGAGAATAAAGAA | Up |
| TAOK3 | 51347 | TAO kinase 3 | NM_016281 | CAGAGACTGTACTACGACAAA | Up |
| TAOK3 | 51347 | TAO kinase 3 | NM_016281 | AAGAAGCAAGTGGCTATCATA | Up |
| APR | 23591 | apoptosis related protein | XM_498424 | CACCGGGATAGATAGAACTAT | Up |
| | | | XM 499554 | | |
| | | | XR_017759 | | |
| | | | XR_017911 | | |
| APR | 23591 | apoptosis related protein | XM_498424 | AAGGAGCTTATTGAGCTATCA | Up |
| | | | XM_499554 | | |
| | | | XR_317759 | | |
| | | | XR_017911 | | |
| APR | 23591 | apoptosis related protein | XM_498424 | ACCGTTTGGGATGGTTGATTA | Up |
| | | | XM_499554 | | |
| | | | XR_017759 | | |
| | | | XR_017911 | | |
| APR | 23591 | apoptosis related protein | XM_498424 | CGGCCCAGCGTTCAAGCCCAA | Up |
| | | | XM_499554 | | |
| | | | XR_017759 | | |
| | | | XR_017911 | | |
| NUAK1 | 9891 | NUAK family, SNF1-like kinase, 1 | NM_014840 | CGGCAGGACTCTTATCTTAAA | Up |
| NUAK1 | 9891 | NUAK family, SNF1-like kinase, 1 | NM_014840 | TTCGATGGTTTCGATCACAAA | Up |
| NUAK1 | 9891 | NUAK family, SNF1-like kinase, 1 | NM_014840 | CAGGTAAATGTCAAGGCACAA | Up |
| PHB | 5245 | prohibitin | NM_002634 | CTGCACTGGGAAGGAAACAAA | Up |
| PHB | 5245 | prohibitin | NM_002634 | CACAGAAGCGGTGGAAGCCAA | Up |
| PHB | 5245 | prohibitin | NM_002634 | CAGGTGAGCGACGACCTTACA | Up |
| YES1 | 7525 | v-yes-1 Yamaguchi sarcoma viral oncogene homolog 1 | NM_005433 | TTGCGACTAGAGGTTAAACTA | Up |
| YES1 | 7525 | v-yes-1 Yamaguchi sarcoma viral oncogene homolog 1 | NM_005433 | AACAGTCAGTATGCAATCTTA | Up |
| YES1 | 7525 | v-yes-1 Yamaguchi sarcoma viral oncogene homolog 1 | NM_005433 | CGGGCTGCTAATATTCTTGTA | Up |
| PKIB | 5570 | protein kinase (cAMP-dependent, catalytic) | NM_032471 | AAGGCTCATAATCTATCAAGA | Up |
| | | inhibitor beta | NM_181794 | | |
| | | | NM_181795 | | |
| PKIB | 5570 | protein kinase (cAMP-dependent, catalytic) | NM_032471 | CAGTAGGCACTTAAGCATTTA | Up |
| | | inhibitor beta | NM_181794 | | |
| | | | NM_181795 | | |
| PKIB | 5570 | protein kinase (cAMP-dependent, catalytic) | NM_032471 | CGCGGCTGGAGTCATGCTATA | Up |
| | | inhibitor beta | NM_181795 | | |
| BIRC3 | 330 | baculoviral IAP repeat-containing 3 | NM_001165 | CAAGAACATGATGTTATTAAA | Down |
| | | | NM_182962 | | |
| BIRC3 | 330 | baculoviral IAP repeat-containing 3 | NM_001165 | CACTACAAACACAATATTCAA | Down |
| | | | NM_182962 | | |
| ARF4 | 378 | ADP-ribosylation factor 4 | NM_001660 | CTGAGACAGTTTGCTAATTTA | Down |
| | | | XM_001132763 | | |
| ARF4 | 378 | ADP-ribosylation factor 4 | NM_001660 | ATCAGTGAAATGACAGATAAA | Down |
| | | | XM_001132763 | | |
| CLK2 | 1196 | CDC-like kinase 2 | NM_001291 | TGCCTTGTACATAATACTATT | Down |
| | | | NM_003993 | | |
| | | | XM_001128256 | | |
| CLK2 | 1196 | CDC-like kinase 2 | NM_001291 | TAGCCGCTCATCTTCGATGAA | Down |
| | | | NM_003993 | | |
| | | | XM_001128256 | | |
| CLK3 | 1198 | CDC-like kinase 3 | NM_001292 | CTGGTTCAACTTCCACGGTCA | Down |
| | | | NM_003992 | | |
| CLK3 | 1198 | CDC-like kinase 3 | NM_001292 | GAGGACTACTATGGACCTTCA | Down |
| | | | NM_003992 | | |
| GSK3B | 2932 | glycogen synthase kinase 3 beta | NM_002093 | CACGTTTGGAAAGAATATTAA | Down |
| GSK3B | 2932 | glycogen synthase kinase 3 beta | NM_002093 | CCCAAATGTCAAACTACCAAA | Down |
| PTGS1 | 5742 | prostaglandin-endoperoxide synthase 1 | NM_000962 | TTGGGCTATAGCACACAGTTA | Down |
| | | (prostaglandin G/H synthase and cyclooxygenase) | NM_080591 | | |
| PTGS1 | 5742 | prostaglandin-endoperoxide synthase 1 | NM_000962 | CACCTACAACTCAGCACATGA | Down |
| | | (prostaglandin G/H synthase and cyclooxygenase) | NM_080591 | | |
| VEGFA | 7422 | vascular endothelial growth factor A | NM_001025366 | CTGGAATTTGATATTCATTGA | Down |
| | | | NM_001025367 | | |
| | | | NM_001025368 | | |
| | | | NM_001025369 | | |
| | | | NM_001025370 | | |
| | | | NM_001033756 | | |
| | | | NM_003376 | | |
| VEGFA | 7422 | vascular endothelial growth factor A | NM_001025366 | AAGCATTTGTTTGTACAAGAT | Down |
| | | | NM_001025367 | | |
| | | | NM_001025368 | | |
| | | | NM_001025369 | | |
| | | | NM_001033756 | | |
| | | | NM_003376 | | |
| PABPC4 | 8761 | Poly(A) binding protein, cytoplasmic 4 (inducible form) | NM_003819 | AACTTTGATGTGATTAAGGGA | Down |
| PABPC4 | 8761 | Poly(A) binding protein, cytoplasmic 4 (inducible form) | NM_003819 | CAGGAGAGAATTAGTCGATAT | Down |
| FADD | 8772 | Fas (TNFRSF6)-associated via death domain | NM_003824 | CTGCAATTCTACAGTTTCTTA | Down |
| FADD | 8772 | Fas (TNFRSF6)-associated via death domain | NM_003824 | CAGCGAGCTGACCGAGCTCAA | Down |
| GMPS | 8833 | guanine monphosphate synthetase | NM_003875 | TACCCTATTACAGAGAGTCAA | Down |
| GMPS | 8833 | guanine monphosphate synthetase | NM_003875 | CCCTGTAGAGGTGGTATTAAA | Down |
| CAMKK2 | 10645 | calcium/calmodulin-dependent protein kinase kinase 2, | NM_006549 | TCAAGTTGGCCTACAATGAAA | Down |
| | | beta | NM_153499 | | |
| | | | NM_153500 | | |
| | | | NM_172214 | | |
| | | | NM_172215 | | |
| | | | NM_172216 | | |
| | | | NM_172226 | | |
| CAMKK2 | 10645 | calcium/calmodulin-dependent protein kinase kinase 2, | NM_006549 | TAGCAGCATGTCCTACGGAAA | Down |
| | | beta | NM_153499 | | |
| | | | NM_153500 | | |
| | | | NM_172216 | | |
| | | | NM_172226 | | |
| CARD8 | 22900 | caspase recruitment domain family, member 8 | NM_014959 | AAGGCGATAGATGATGAGGAA | Down |
| CARD8 | 22900 | caspase recruitment domain family, member 8 | NM_014959 | GAGATCGAAGAAGATTATAAA | Down |
| ITPKC | 80271 | inositol 1,4,5-trisphosphate 3-kinase C | NM_025194 | CAGAAGGAGCCTGTCCCTCAA | Down |
| ITPKC | 80271 | inositol 1,4,5-trisphosphate 3-kinase C | NM_025194 | CAGGACCTATCTGGAAGAGGA | Down |
| CKB | 1152 | creatine kinase, brain | NM_001823 | GCCCTGCTGCTTCCTAACTTA | Up |
| CKB | 1152 | creatine kinase, brain | NM_001823 | GCGGGCAGGTGTGCATATCAA | Up |
| DGUOK | 1716 | deoxyguanosine kinase | NM_080915 | CCGGATCACATTACATGGCTT | Up |
| | | | NM_080916 | | |
| | | | NM_080917 | | |
| DGUOK | 1716 | deoxyguanosine kinase | NM_080915 | CAGCTGCATGGCCAACACGAA | Up |
| | | | NM_080916 | | |
| | | | NM_080917 | | |
| ITGA5 | 3678 | integrin, alpha 5 (fibronectin receptor, alpha | NM_002205 | TGGGCCAACAAAGAACACTAA | Up |
| | | polypeptide) | | | |
| ITGA5 | 3678 | integrin, alpha 5 (fibronectin receptor, alpha | NM_002205 | CAGGGTCTACGTCTACCTGCA | Up |
| | | polypeptide) | | | |
| LTBR | 4055 | lymphotoxin beta receptor (TNFR superfamily, member 3) | NM_002342 | AAAGGGAGTCATTAACAACTA | Up |
| LTBR | 4055 | lymphotoxin beta receptor (TNFR superfamily, member 3) | NM_002342 | CACGGTACCAATGGCATTCAT | Up |
| NOS2A | 4843 | nitric oxide synthase 2A (inducible, hepatocytes) | NM_000625 | ATCGAATTTGTCAACCAATAT | Up |
| | | | NM_153292 | | |
| NOS2A | 4843 | nitric oxide synthase 2A (inducible, hepatocytes) | NM_000625 | CCGGGACTTCTGTGACGTCCA | Up |
| | | | NM_153292 | | |
| PHKA1 | 5255 | phosphorylase kinase, alpha 1 (muscle) | NM_002637 | AAAGATAGTCGTCAAGGTCAA | Up |
| PHKA1 | 5255 | phosphorylase kinase, alpha 1 (muscle) | NM_002637 | CCCAATCGTCTGTACTATGAA | Up |
| RBBP6 | 5930 | retinoblastoma binding protein 6 | NM_006910 | TTGGAGGTGTTAAATCTACAA | Up |
| | | | NM_018703 | | |
| | | | NM_032626 | | |
| RBBP6 | 5930 | retinoblastoma binding protein 6 | NM_006910 | TGCGCAGACGAAAGAAGAATA | Up |
| | | | NM_018703 | | |
| | | | NM_032626 | | |
| TK1 | 7083 | thymidine kinase 1, soluble | NM_003258 | CCGGGAAGCCGCCTATACCAA | Up |
| TK1 | 7083 | thymidine kinase 1, soluble | NM_003258 | CAGGAACAACAGCATCTTTCA | Up |
| PIP5K2B | 8396 | phosphatidylinositol-4-phosphate 5-kinase, type II, | NM_003559 | CAAGGACTTGCCAACATTCAA | Up |
| | | beta | NM_138687 | | |
| PIP5K2B | 8396 | phosphatidylinositol-4-phosphate 5-kinase, type II, | NM_003559 | CACGCGTTTCCTCACCACCTA | Up |
| | | beta | NM_138687 | | |
| TNFRSF18 | 8784 | tumor necrosis factor receptor superfamily, member 18 | NM_004195 | CCCTGGGAACAAGACCCACAA | Up |
| | | | NM_148902 | | |
| TNFRSF18 | 8784 | tumor necrosis factor receptor superfamily, member 18 | NM_004195 | CAGCAGAAGTGGGTGCAGGAA | Up |
| | | | NM_148901 | | |
| | | | NM_148902 | | |
| KIF3B | 9371 | kinesin family member 3B | NM_004798 | AACGCTAAGGTGGGTAGCCTA | Up |
| KIF3B | 9371 | kinesin family member 3B | NM_004798 | CAGAAATGCATGGGTAAGGTA | Up |
| TRAIP | 10293 | TRAF interacting protein | NM_005879 | CAGCAGGATGAGACCAAACAA | Up |
| TRAIP | 10293 | TRAF interacting protein | NM_005879 | CCGGCTCAGGAGCAAGATGAA | Up |
| STK33 | 65975 | serine/threonine kinase 33 | NM_030906 | TCCATAAGTGACTGTGCTAAA | Up |
| STK33 | 65975 | serine/threonine kinase 33 | NM_030906 | GAGCATAGGCGTCGTAATGTA | Up |
| MAPKAP1 | 79109 | mitogen-activated protein kinase associated protein 1 | NM_001006617 | AAGGGTCATGTAGGTACAACA | Up |
| | | | NM_001006618 | | |
| | | | NM_001006619 | | |
| | | | NM_001006620 | | |
| | | | NM_001006621 | | |
| | | | NM_024117 | | |
| MAPKAP1 | 79109 | mitogen-activated protein kinase associated protein 1 | NM_001006617 | CTCCCTTATTCAGGTGGACAA | Up |
| | | | NM_001006618 | | |
| | | | NM_001006619 | | |
| | | | NM_001006620 | | |
| | | | NM_001006621 | | |
| | | | NM_024117 | | |
| PANK3 | 79646 | pantothenate kinase 3 | NM_024594 | TTGCAGTACTGTATGCTCAAA | Up |
| PANK3 | 79646 | pantothenate kinase 3 | NM_024594 | CTGCTGAATGTTGCCATATTA | Up |
| UCK1 | 83549 | uridine-cytidine kinase 1 | NM_031432 | CCCACGAGGAGTGGACAATAT | Up |
| UCK1 | 83549 | uridine-cytidine kinase 1 | NM_031432 | AAGGCTGGTTGTGGCCTACAA | Up |
| PIK3AP1 | 118788 | phosphoinositide-3-kinase adaptor protein 1 | NM_152309 | AAGTACGACTGTAGTTATCTA | Up |
| PIK3AP1 | 118788 | phosphoinositide-3-kinase adaptor protein 1 | NM_152309 | TCCCATGGGATTATTCTCTAT | Up |

Table 1b

| | | | | | | | Infectivity |
|---|---|---|---|---|---|---|---|
| NCBI | Entrez | | | | | | phenotype |
| gene | Gene | EC | | mRNA | siRNA | | upon |
| symbol | Id | number | Gene description | Accessions | Target Sequence | Product Name | knockdown |
| ADCY8 | 114 | 4.6.1.1 | adenylate cyclase 8 (brain) | NM_001115 | CACCGGCATTGAGGTAGTGAT | Hs_ADCY8_5 | down |
| ADCY8 | 114 | 4.6.1.1 | adenylate cyclase 8 (brain) | NM_001115 | CAGGCCGCCTTTGAGTCTTTA | Hs_ADCY8_8 | down |
| ADCY9 | 115 | 4.6.1.1 | adenylate cyclase 9 | NM_001116 | CTGGGCATGAGGAGGTTTAAA | Hs_ADCY9_3 | down |
| AMPD3 | 272 | 3.5.4.6 | adenosine monophosphate deaminase 3 | NM_000480 | CGGGATCACTTTGGAGGACTA | Hs_AMPD3_1 | down |
| | | | | NM_001025389 | | | |
| | | | | NM_001025390 | | | |
| | | | | NM_001172430 | | | |
| AMPD3 | 272 | 3.5.4.6 | adenosine monophosphate deaminase 3 | NM_000480 | CAACAGTTTGTTCCTCGAATA | Hs_AMPD3_9 | down |
| | | | | NM_001025389 | | | |
| | | | | NM_001025390 | | | |
| | | | | NM_001172430 | | | |
| | | | | NM_001172431 | | | |
| ENPP1 | 5167 | 3.1.4.1 | ectonucleotide pyrophosphatase/ | NM_006208 | TGGGCAAACAGTAGACTTATA | Hs_ENPP1_2 | down |
| | | 3.6.1.9 | phosphodiesterase 1 | | | | |
| ENPP1 | 5167 | 3.1.4.1 | ectonucleotide pyrophosphatase/ | NM_006208 | AAGCATGAAALTTTACCCTAT | Hs_ENPP1_4 | down |
| | | 3.6.1.9 | phosphodiesterase 1 | | | | |
| ENPP3 | 5169 | 3.1.4.1 | ectonucleotide pyrophosphatase/ | NM_005021 | CTGGCTGTTAGGAGTAAATCA | Hs_ENPP3_1 | down |
| | | 3.6.1.9 | phosphodiesterase 3 | | | | |
| ENPP3 | 5169 | 3.1.4.1 | ectonucleotide pyrophosphatase/ | NM_005021 | TAGCAATTTGGTACCTATGTA | Hs_ENPP3_2 | down |
| | | 3.6.1.9 | phosphodiesterase 3 | | | | |
| ENPP5 | 59084 | 3.1.— | ectonucleotide pyrophosphatase/ | NM_021572 | AAGCATTTAATTCACAGTCAA | Hs_ENPP5_1 | down |
| | | | phosphodiesterase 5 (putative function) | | | | |
| ENPP6 | 133121 | 3.1.— | ectonucleotide pyrophosphatase/ | NM_153343 | TTGGATAATTCTATACATAAA | Hs_ENPP6_1 | down |
| | | | phosphodiesterase 6 | | | | |
| ENPP6 | 133121 | 3.1.— | ectonucleotide pyrophosphatase/ | NM_153343 | CAGGGTGATGTGCATGCTGAA | Hs_ENPP6_2 | down |
| | | | phosphodiesterase 6 | | | | |
| ENTPD4 | 9583 | 3.6.1.6 | ectonucleoside triphosphate | NM_004901 | CAGCAGGAAGAAGTAGCTAAA | Hs_ENTPD4_1 | down |
| | | | diphosphohydrolase 4 | | | | |
| ENTPD4 | 9583 | 3.6.1.6 | ectonucleoside triphosphate | NM_001249 | CAGCAGAAAGCTATTCTGGAA | Hs_ENTPD4_2 | down |
| | | | diphosphohydrolase 4 | | | | |
| GMPR2 | 51292 | 1.7.1.7 | guanosine monophosphate reductase 2 | NM_001002000 | TCCCAAGGCACCAGTACTCTA | Hs_GMPR2_11 | down |
| | | | | NM_001002001 | | | |
| | | | | NM_001002002 | | | |
| | | | | NM_016576 | | | |
| GMPR2 | 51292 | 1.7.1.7 | guanosine monophosphate reductase 2 | NM_001002000 | TTGGACCTTCACATATCTAAA | Hs_GMPR2_12 | down |
| | | | | NM_001002001 | | | |
| | | | | NM_001002002 | | | |
| | | | | NM_016576 | | | |
| GMPS | 8833 | 6.3.5.2 | guanine monphosphate synthetase | NM_003875 | AACAGAGAACTTGAGTGTATT | Hs_GMPS_1 | down |
| GMPS | 8833 | 6.3.5.2 | guanine monphosphate synthetase | NM_003875 | AAGAATTTCCTTTATGATATA | Hs_GMPS_2 | down |
| GUK1 | 2987 | 2.7.4.8 | guanylate kinase 1 | NM_000858 | CCCGGCGAGGAGAACGGCAAA | Hs_GUK1_6 | down |
| | | | | NM_001159390 | | | |
| | | | | NM_001159391 | | | |
| HPRT1 | 3251 | 2.4.2.8 | hypoxanthine phosphoribosyltransferase 1 | NM_000194 | CCCACGAAGTGTTGGATATAA | Hs_HPRT1_6 | down |
| HPRT1 | 3251 | 2.4.2.8 | hypoxanthine phosphoribosyltransferase 1 | NM_000194 | CCACAGCACTATTGAGTGAAA | Hs_HPRT1_9 | down |
| IMPDH1 | 3614 | 1.1.1.205 | IMP (inosine 5′-monophosphate) | NM_000883 | CAGCAGCAGCCAGAAACGATA | Hs_IMPDH1_6 | down |
| | | | dehydrogenase 1 | NM_001102605 | | | |
| | | | | NM_001142573 | | | |
| | | | | NM_001142574 | | | |
| | | | | NM_001142575 | | | |
| | | | | NM_001142576 | | | |
| | | | | NM_183243 | | | |
| IMPDH1 | 3614 | 1.1.1.205 | IMP (inosine 5′-monophosphate) | NM_000883 | CAGGCGGGCGTCGACGTCATA | Hs_IMPDH1_7 | down |
| | | | dehydrogenase 1 | NM_001102605 | | | |
| | | | | NM_001142573 | | | |
| | | | | NM_001142574 | | | |
| | | | | NM_001142575 | | | |
| | | | | NM_001142576 | | | |
| | | | | NM_183243 | | | |
| IMPDH2 | 3615 | 1.1.1.205 | IMP (inosine 5′-monophosphate) | NM_000884 | TGGGATCCGGCTAAAGAAATA | Hs_IMPDH2_6 | down |
| | | | dehydrogenase 2 | | | | |
| IMPDH2 | 3615 | 1.1.1.205 | IMP (inosine 5′-monophosphate) | NM_000884 | AATGTGGGTCATATTGCGAAA | Hs_IMPDH2_8 | down |
| | | | dehydrogenase 2 | | | | |
| ITPA | 3704 | 3.6.1.19 | inosine triphosphatase (nucleoside | NM_033453 | CTGGAGAAGTTAAAGCCTGAA | Hs_ITPA_2 | down |
| | | | triphosphate pyrophosphatase) | | | | |
| ITPA | 3704 | 3.6.1.19 | inosine triphosphatase (nucleoside | NM_033453 | GAGGAGGTCGTTCAGATTCTA | Hs_ITPA_3 | down |
| | | | triphosphate pyrophosphatase) | | | | |
| NME1 | 4830 | 2.7.4.6 | non-metastatic cells 1, protein (NM23A) | NM_000269 | TTCCGCCTTGTTGGTCTGAAA | Hs_NME1_5 | down |
| | | | expressed in | | | | |
| NME1 | 4830 | 2.7.4.6 | non-metastatic cells 1, protein (NM23A) | NM_000269 | TCCGAAGATCTTCTCAAGGAA | Hs_NME1_8 | down |
| | | | expressed in | | | | |
| NME2 | 4831 | 2.7.4.6 | non-metastatic cells 2, protein (NM23B) | NM_002512 | CAGCACTACATTGACCTGAAA | Hs_NME2_22 | down |
| | | | expressed in | | | | |
| NME2 | 4831 | 2.7.4.6 | non-metastatic cells 2, protein (NM23B) | NM_002512 | TAGAGCATATTTGCCAATAAA | Hs_NME2_3 | down |
| | | | expressed in | | | | |
| NME2 | 4831 | 2.7.4.6 | non-metastatic cells 2, protein (NM23B) | NM_002512 | TGGGCTGGTGAAGTACATGAA | Hs_NME2_6 | down |
| | | | expressed in | | | | |
| NME2P1 | 283458 | 2.7.4.6 | non-metastatic cells 2, protein (NM23B) | NR_001577 | CAGGTTGGCAGGAACATCATT | Hs_NME2P1_2 | down |
| | | | expressed in, pseudogene 1 | | | | |
| NME2P1 | 283458 | 2.7.4.6 | non-metastatic cells 2, protein (NM23B) | NR_001577 | CCTGGTGGGCAAGATCATCAA | Hs_NME2P1_4 | down |
| | | | expressed in, pseudogene 1 | | | | |
| NME3 | 4832 | 2.7.4.6 | non-metastatic cells 3, protein expressed in | NM_002513 | CTGCATCGAGGTTGGCAAGAA | Hs_NME3_3 | down |
| NME3 | 4832 | 2.7.4.6 | non-metastatic cells 3, protein expressed in | NM_002513 | ACGGCCGCCTTGTCAAGTATA | Hs_NME3_5 | down |
| NPR2 | 4882 | 4.6.1.2 | natriuretic peptide receptor B/ | NM_000907 | ACCCAACTGAATGAAGAGCTA | Hs_NPR2_2 | down |
| | | | guanylate cyclase B | NM_003995 | | | |
| | | | (atrionatriuretic peptide receptor B) | | | | |
| NPR2 | 4882 | 4.6.1.2 | natriuretic peptide receptor B/ | NM_000907 | CAGCTCAGCCCTGTACATATA | Hs_NPR2_8 | down |
| | | | guanylate cyclase B | NM_003995 | | | |
| | | | (atrionatriuretic peptide receptor B) | | | | |
| PAPSS2 | 9060 | 2.7.7.4 | 3′-phosphoadenosine 5′-phosphosulfate | NM_001015880 | ATGGAAGGTCCTGACAGATTA | Hs_PAPSS2_1 | down |
| | | | synthase 2 | NM_004670 | | | |
| PAPSS2 | 9060 | 2.7.7.4 | 3′-phosphoadenosine 5′-phosphosulfate | NM_001015880 | AACATTGTACCCTATACTATA | | down |
| | | | synthase 2 | NM_004670 | | | |
| PDE1A | 5136 | 3.1.4.17 | phosphodiesterase 1A, calmodulin-dependent | NM_005019 | ACAGAGTGCTATAACTATAAA | Hs_PDE1A_2 | down |
| PDE1A | 5136 | 3.1.4.17 | phosphodiesterase 1A, calmodulin-dependent | NM_005019 | ACACAGAGTGCTATAACTATA | Hs_PDE1A_4 | down |
| PDE2A | 5138 | 3.1.4.17 | phosphodiesterase 2A, cGMP-stimulated | NM_002599 | ACCGCTTTGTACATGAGAATA | Hs_PDE2A_3 | down |
| PDE2A | 5138 | 3.1.4.17 | phosphodiesterase 2A, cGMP-stimulated | NM_002599 | ATCGCGGAGCTGATCTACAAA | Hs_PDE2A_4 | down |
| PDE4A | 5141 | 3.1.4.17 | phosphodiesterase 4A, cAMP-specific | NM_006202 | TTCGATCTTGTCTCCAATTAA | Hs_PDE4A_2 | down |
| | | | (phosphodiesterase E2 dunce homolog, | | | | |
| | | | Drosophila) | | | | |
| PDE4A | 5141 | 3.1.4.17 | phosphodiesterase 4A, cAMP-specific | NM_006202 | ATGGGAAGTCGTGTCATCCTA | Hs_PDE4A_4 | down |
| | | | (phosphodiesterase E2 dunce homolog, | | | | |
| | | | Drosophila) | | | | |
| PDE4D | 5144 | 3.1.4.17 | phosphodiesterase 4D, cAMP-specific | NM_006203 | TACCCACTAATGGACAATCAA | Hs_PDE4D 3 | down |
| | | | (phosphodiesterase E3 dunce homolog, | | | | |
| | | | Drosophila) | | | | |
| PDE4D | 5144 | 3.1.4.17 | phosphodiesterase 4D, cAMP-specific | NM_006203 | CAGGTAGTTAAGTTAGGGTTA | Hs_PDE4D_4 | down |
| | | | (phosphodiesterase E3 dunce homolog, | | | | |
| | | | Drosophila) | | | | |
| PDE6B | 5158 | 3.1.4.35 | phosphodiesterase 6B, cGMP-specific, rod, | NM_000283 | CACGCTGCTCATGACCGGCAA | Hs_PDE6B_2 | down |
| | | | beta (congenital stationary night | | | | |
| | | | blindness 3, autosomal dominant) | | | | |
| PDE6B | 5158 | 3.1.4.35 | phosphodiesterase 6B, cGMP-specific, rod, | NM_000283 | GCCCACCACATTTGACATCTA | Hs_PDE6B_4 | down |
| | | | beta (congenital stationary night | | | | |
| | | | blindness 3, autosomal dominant) | | | | |
| PDE6C | 5146 | 3.1.4.35 | phosphodiesterase 6C, cGMP-specific, cone, | NM_006204 | CACGGTTAGATCATATCTGAA | Hs_PDE6C_1 | down |
| | | | alpha prime | | | | |
| PDE6C | 5146 | 3.1.4.35 | phosphodiesterase 6C, cGMP-specific, cone, | NM_006204 | AAGGTCGTCCTTTCTGAACAA | Hs_PDE6C_3 | down |
| | | | alpha prime | | | | |
| PDE6G | 5148 | 3.1.4.17 | phosphodiesterase 6G, cGMP-specific, rod, | NM_002602 | CTCCGTCAGCCTCACCATGAA | Hs_PDE6G_1 | down |
| | | | gamma | | | | |
| PDE6G | 5148 | 3.1.4.17 | phosphodiesterase 6G, cGMP-specific, rod, | NM_002602 | GACGACATCCCTGGAATGGAA | Hs_PDE6G_3 | down |
| | | | gamma | | | | |
| PDE8B | 8622 | 3.1.4.17 | phosphodiesterase 8B | NM_003719 | CACAAGATTCATCGTGATTCA | Hs_PDE8B_2 | down |
| PDE8B | 8622 | 3.1.4.17 | phosphodiesterase 8B | NM_003719 | TTGGAAGCCATTACGCATAAA | Hs_PDE8B_4 | down |
| PPAT | 5471 | 2.4.2.14 | phosphoribosyl pyrophosphate | NM_002703 | CACAGAGATGTTATTTATGCA | Hs_PPAT_2 | down |
| | | | amidotransferase | | | | |
| PPAT | 5471 | 2.4.2.14 | phosphoribosyl pyrophosphate | NM_002703 | CAGTACGAGATCCTTATGGAA | Hs_PPAT_5 | down |
| | | | amidotransferase | | | | |
| PRPS2 | 5634 | 2.7.6.1 | phosphoribosyl pyrophosphate synthetase 2 | NM_001039091 | AGGCGACAACTTTCAAGTATA | Hs_PRPS2_1 | down |
| | | | | NM_002765 | | | |
| PRPS2 | 5634 | 2.7.6.1 | phosphoribosyl pyrophosphate synthetase 2 | NM_001039091 | AACCCTGTATAGAATTAATTA | Hs_PRPS2_4 | down |
| | | | | NM_002765 | | | |

1-42. (canceled)

43. A pharmaceutical composition comprising at least one inhibitor of a microorganism selected from the family Chlamydiaceae, optionally together with pharmaceutically acceptable carriers, adjuvants, diluents or/and additives, wherein the inhibitor is selected from compounds capable of inhibiting the nucleotide metabolism, in particular nucleotide metabolism essential for chlamydial growth, propagation or/and infection.

44. The pharmaceutical composition as claimed in claim 43, wherein inhibition of the nucleotide metabolism includes
(a) inhibition of the activity of GMP synthase, in particular GMP synthase EC 6.3.5.2, more particular GMP synthase described by genbank entry NM_{—}003875, or
(b) inhibition of the activity of IMP dehydrogenase 2, in particular IMP dehydrogenase 2 EC 1.1.1.205, more particular IMP dehydrogenase 2 described by genbank entry NM_{—}000884.

45. The pharmaceutical composition as claimed in claim 43, wherein inhibition comprises inhibition of growth or/and propagation of the microorganism selected from the family Chlamydiaceae.

46. The pharmaceutical composition as claimed in claim 43, wherein inhibition comprises inhibition of the interaction of the microorganism with the host cell.

47. The pharmaceutical composition as claimed in claim 43, wherein inhibition comprises
(i) reduction of the number of EB that infected the host cell, or/and
(ii) reduction of the number of RB inside the host cell.

48. The pharmaceutical composition as claimed in claim 43, wherein the at least one inhibitor of the microorganism is selected from the group of nucleic acids, nucleic acid analogues such as ribozymes, peptides, polypeptides, and antibodies, wherein the nucleic acid encodes a GMP synthase or a IMP dehydrogenase 2, or/and a fragment thereof, and wherein the antibody is directed against a GMP synthase or a IMP dehydrogenase 2 or/and a fragment thereof.

49. The pharmaceutical composition as claimed in claim 48, wherein the nucleic acid is RNA, and wherein the RNA molecule preferably is a double-stranded RNA molecule, more preferably a double-stranded siRNA molecule with or without a single-stranded overhang alone at one end or at both ends, wherein the siRNA molecule is preferably directed against a sequence selected from nucleic acid sequences encoding a GMP synthase or a IMP dehydrogenase 2 and fragments thereof.

50. The pharmaceutical composition as claimed in claim 48, wherein the nucleic acid has a length of at least 15, preferably at least 17, more preferably at least 19, most preferably at least 21 nucleotides, or/and has a length of at the maximum 29, preferably at the maximum 27, more preferably at the maximum 25, especially more preferably at the maximum 23, most preferably at the maximum 21 nucleotides.

51. The pharmaceutical composition as claimed in claim 43 for use in the treatment or/and prophylaxis of an infection with a microorganism selected from the family Chlamydiaceae.

52. A method for the treatment or/and prophylaxis of an infection with a microorganism selected from the family Chlamydiaceae, comprising administering a pharmaceutical composition of claim 43 to a subject in need thereof.
